(19) United States
(12) Patent Application Publication
(10) Pub. No.: US 2003/0125252 A1
(43) Pub. Date: Jul. 3, 2003

(54) Compositions and methods for affecting osteogenesis

(76) Inventors: T. Michael Underhill, Ontario (CA); Arthur V. Sampaio, Ontario (CA); Andrea D. Weston, Ontario (CA)

Correspondence Address: MYERS BIGEL SIBLEY & SAJOVEC, PO BOX 37428, RALEIGH, NC, 27627, US

(21) Appl. No.: 10/221,602

(22) PCT Filed: Mar. 13, 2001
(86) PCT No.: PCT/CA01/00317

(51) Int. Cl. A61K 38/18; A61K 38/29; A61K 31/66; A61K 31/56; A61K 31/353; A61K 31/557; A61K 31/12; A61K 31/122; A61K 33/10; A61K 33/14

(52) U.S. Cl. 514/12; 514/102; 514/456; 514/573; 514/682; 514/171; 424/673; 424/687

(57) ABSTRACT

The invention relates to compositions for promoting and inhibiting osteogenesis and to methods for treating bone abnormalities resulting from injury, toxicity or disease and for ex vivo bone tissue engineering.

### FIELD OF THE INVENTION

The invention relates to compositions for affecting osteogenesis in vitro and in vivo. In particular, the invention relates to compositions for stimulating and inhibiting osteogenesis and methods for the use thereof for treating bone abnormalities resulting from injury, toxicity or disease and for ex vivo bone tissue engineering.

### BACKGROUND OF THE INVENTION

Throughout this application, various references are cited in parentheses to describe more fully the state of the art to which this invention pertains. Full bibliographic information for each citation is found at the end of the specification, immediately preceding the claims. The disclosure of these references are hereby incorporated by reference into the present disclosure.

The active form of vitamin D, 1α,25-dihydroxy vitamin-D₃ (1,25 VD₃), functions in the maintenance of calcium homeostasis and is important in elevating blood calcium levels by increasing uptake from the intestinal lumen, limiting excretion through the kidney and releasing Ca²⁺ through resorption of bone (1). While many of the effects of 1,25 VD₃ on bone are thought to be secondary to its action on plasma Ca²⁺, several studies have demonstrated involvement of 1,25 VD₃ in osteoblast function (1,2). Exposure of cultured osteoblasts to 1,25 VD₃ leads to changes in phenotype which are dependent upon the stage at which the cells are treated (3). Exposure of preosteoblasts to 1,25 VD₃ inhibits deposition of an extracellular matrix and its subsequent mineralization. Paradoxically, at later stages, 1,25 VD₃ exposure stimulates osteoblastic maturation and enhances matrix synthesis and calcium deposition.

Vitamin D₃ functions, in part, through activation of vitamin D₃ receptor (VDR), a member of the nuclear receptor superfamily (4,5). VDR is expressed abundantly in the kidney, bone, intestine and skin and is expressed at lower levels in a number of other tissues (6-9). More recently, an isoform of VDR has been identified which may be important in mediating some of the tissue-specific actions of 1,25 VD₃ (10). VDR regulates gene expression by interacting with DNA either as a homodimer or as a heterodimer, typically with a retinoid-X-receptor (RXR) (1,2,5). VDR has also been shown to interact with other members of the steroid hormone superfamily of receptors in vitro, including retinoic acid receptors (RAR). These interactions may also contribute to VDR function (11,12). VDR-null mutants are phenotypically normal at birth, but after weaning develop a disease similar to 1,25 VD₃-dependent rickets type II, which is indicative of a role for VDR in bone formation during bone remodeling (13,14).

Retinoids also affect skeletal development and homeostasis. Studies report conflicting effects of retinoids on osteoblast development. During embryogenesis, exogenous retinoids can inhibit skeletal development through inhibition of chondrogenesis, subsequently leading to an inadequate cartilaginous template to support bone formation (15). Hypervitaminosis-A has been reported to inhibit bone formation in vivo (16,17). Post-natal exposure to vitamin-A affects osteogenesis causing bone lesions, and thinned bone collars, and may contribute to osteoporosis. Exposure of preosteoblastic cells or differentiating osteoblasts to vitamin-A inhibits matrix synthesis and mineralization. These actions of vitamin-A are mediated predominantly through the vitamin-A metabolite retinoic acid (RA) and its association with receptors for either all-trans and 9-cis RA, the retinoic acid receptors (RAR) or 9-cis RA the retinoid-X-receptors (RXR) (18). The RARs function as heterodimers in association with RXR partners, but may also interact with VDR or thyroid hormone receptors in certain cell types to regulate gene expression (19). VDR, RARα and RXRα are co-expressed in osteoblastic cells and are likely important in mediating the effects of their respective ligands on the osteoblast phenotype.

Studies have also reported that the addition of retinoids to osteoblast cultures stimulates bone formation or enhances the expression of gene indicative of enhanced osteoblast function (40, 42, 43, 44, 46, 47, 61, 49, 50, 52, 56, 60). However, other reports have shown that addition of RA can inhibit bone formation in vitro (41, 51, 58). Some of the effects of RA on bone formation may be dependent on the stage of osteoblast differentiation and the species.

Some reports have shown that RA can stimulate osteoporosis in vivo. One such study was performed in rats (59) however, in this model they found the primary mechanism for reduced bone mineral density was an increase in bone resorption through activation of osteoclasts. In keeping with these results, RA has been reported to stimulate osteoclast activity (45, 53, 54, 55, 58) which would result in increased bone resorption manifesting in osteoporosis. An additional report has shown that intermittent RA treatment can stimulate bone formation in rats (57) while a radiographic study performed on humans treated with 13-cis RA for acne showed no evidence of an effect of RA on bone mineral density (48).

Although the studies have indicated a general involvement of Vitamin D and retinoic acid receptors on osteoblastic cells, the nature of the relationship has not been previously ellucidated with respect to bone cell development. No one has previously demonstrated a direct effect on retinoic acid receptor activity and vitamin D function and consequently, a resultant effect on osteogenesis. Furthermore, none of the literature consistently or definitively demonstrates that inhibition of retinoid signaling, in any manner, stimulates bone formation.

### SUMMARY OF THE INVENTION

There is now demonstrated a novel functional requirement for RAR-mediated signaling in vitamin D action on osteoblasts. Treatment of the MC3T3-E1 preosteoblastic cell line with vitamin D₃ inhibited mineralization and stimulated programmed cell death. An RAR-selective agonist potentiated these effects, whereas an RAR-selective antagonist reversed the effects of vitamin D on both cell death and to a lesser extent, on bone nodule formation. The antagonist also stimulated mineralization and expression of osteocalcin. Vitamin D-induced cell death was inhibited by expression of a dominant-negative RAR. RAR antagonists have also been demonstrated to stimulate bone formation. These results demonstrate that inhibition of osteogenesis by vitamin D involves stimulation of apoptosis in preosteoblasts and that vitamin D and RAR-mediated signaling pathways cooperate to regulate expression of the osteoblastic phenotype.

The results indicate that RAR activity has a direct role in reversing vitamin D induced cell death. This provides a basis for the development of therapeutic compositions and uses of such compositions to treat disorders involving inappropriate bone formation.

In one aspect, the therapeutic compositions and methods using such are based on the inhibition of RAR activity. Thus compositions may include any RAR antagonist or agent having RAR antagonist activity. As such, the compositions may include antisense RAR oligonucleotides which down-regulate or inhibit RAR activity.

The present invention provides therapeutic compositions and methods for the treatment of disorders involving abnormal bone formation and associated abnormal skeletal development resulting from disease, trauma, vitamin D toxicity and hypervitaminosis A.

In accordance with a first embodiment, the present invention provides a pharmaceutical composition comprising an effective amount of an RAR antagonist and, optionally, a pharmaceutically acceptable carrier for the promotion of osteogenesis.

In accordance with another embodiment of the present invention is a pharmaceutical composition comprising an effective amount of an RAR antagonist and, optionally, a pharmaceutically acceptable carrier for the treatment of vitamin D toxicity.

In accordance with another embodiment of the present invention is a pharmaceutical composition comprising an effective amount of an RAR antagonist and, optionally, a pharmaceutically acceptable carrier for the treatment of adynamic bone disease.

In accordance with another embodiment of the present invention is a pharmaceutical composition comprising an effective amount of an RAR antagonist and, optionally, a pharmaceutically acceptable carrier for the regulation of VDR transcriptional activity in vivo and in vitro.

The present invention additionally relates to the use of RAR antagonists for blocking all or some RAR receptor sites in biological systems, including mammals, to prevent or dimish action of RAR agonists on said receptor sites. More particularly, the present invention relates to the use of RAR antagonists for (a) the prevention and (b) the treatment of retinoid (including vitamin A or vitamin A precursor) chronic or acute toxicity and side effects of retinoid therapy. In this aspect of the present invention, there is provided a method of treating a pathological condition in a mammal. The conditions treated are associated with a retinoic acid receptor activity. This method involves administering to the mammal a retinoid antagonist or analogue thereof capable of binding to one of the following retinoic acid receptor subtypes: RARα, RARβ and RARλ. The antagonist is administered in an amount pharmaceutically effective to provide a therapeutic benefit against the pathological condition in the mammal.

RAR antagonists for use in the present invention are characterized by having a stimulating effect on bone formation and as a result on bone development in a vertebrate. RAR antagonists may be defined as any chemical that binds to one or more of the RAR subtypes with a Kd of less than 1 micromolar. Conventionally, a RAR antagonist is a chemical agent that inhibits the activity of an RAR agonist. Thus the activity of a receptor antagonist is conventionally measured by virtue of its ability to inhibit the activity of an agonist.

In accordance with another embodiment of the present invention, is the use of an RAR antagonist for inhibiting apoptosis in osteoblastic cells exposed to vitamin D.

In accordance with another embodiment of the present invention, is the use of an RAR antagonist for promoting osteoblast differentiation leading to the stimulation of mineralization and expression of certain genes such as osteocalcin and bone sialoprotein in osteoblastic cells.

In accordance with a further embodiment, the invention provides a method for stimulating osteogenesis in a vertebrate, the method comprising administering to the vertebrate an effective osteogenesis stimulating amount of an RAR antagonist.

In accordance with a further embodiment, the invention provides a method for treating damaged bone in a subject, comprising administering to the subject an effective amount of an RAR antagonist, wherein the RAR antagonist stimulates bone repair and formation.

In accordance with a further embodiment, the invention provides a method for enhancing osseous integration of orthopedic or dental implants in a subject comprising administering to the subject an effective amount of an RAR antagonist.

The methods may involve providing systemic or local administration of the selected RAR antagonist.

In accordance with a further embodiment, the invention provides a method for treating bone associated disorders in a subject, comprising administering to the subject cells selected from the group consisting of osteoblastic cells, preosteoblastic cells, skeletal progenitor cells derived from bone, bone marrow or blood, and mixtures thereof, treated with an effective amount of an RAR antagonist.

According to one embodiment of the invention, there is provided a composition for inducing osteogenesis and associated skeletal development in a vertebrate, the composition comprising:

a RAR antagonist; and

a pharmaceutically acceptable carrier.

According to another embodiment of the invention, there is provided a morphogenetic device for implantation at a bone site in a vertebrate, the device comprising:

an implantable biocompatible carrier; and

a RAR antagonist dispersed within or on said carrier.

According to yet another embodiment of the invention, there is provided the use of a composition comprising a RAR antagonist and a pharmaceutically acceptable carrier, for inducing osteogenesis in vitro.

In one aspect of the invention, the composition may comprise antisense oligonucleotides which down-regulate or inhibit RAR activity.

According to yet another embodiment of the invention, there is provided a method for stimulating mineralization of osteoblastic cells comprising contacting an osteoblastic cell with a RAR antagonist in vitro.

According to another embodiment of the invention, there is provided an implantable prosthetic device for repairing bone-associated orthopedic defects, injuries or anomalies in a vertebrate, the device comprising:

a prosthetic implant having a surface region implantable adjacent to or within a bone tissue.

a RAR antagonist composition disposed on the surface region in an amount sufficient to promote enhanced bone mineralization and bone formation on the surface.

According to another embodiment of the invention, there is provided a method for promoting in vivo integration of an implantable prosthetic device into a target bone tissue of a vertebrate, the method comprising the steps of:

providing on a surface of the prosthetic device a composition comprising a RAR antagonist and a pharmaceutically acceptable carrier and

implanting the device in a vertebrate at a site where the target bone tissue and the surface of the prosthetic device are maintained at least partially in contact for a time sufficient to permit tissue growth between the target bone tissue and the device.

According to yet another embodiment of the invention, there is provided a method for promoting natural bone formation at a site of skeletal surgery in a vertebrate, the method comprising the steps of delivering a RAR antagonist composition to the site of the skeletal surgery whereby such delivery indirectly promotes the formation of new bone tissue.

According to another embodiment of the invention, there is provided a method for repairing large segmental skeletal gaps and non-union fractures arising from trauma or surgery in a vertebrate, the method comprising delivering a RAR antagonist composition to the site of the segmental skeletal gap or non-union fracture whereby such delivery promotes the formation of new bone tissue formation.

According to yet another embodiment of the invention, there is provided a method for aiding the attachment of an implantable prosthesis to a bone site and for maintaining the long term stability of the prosthesis in a vertebrate, the method comprising coating selected regions of an implantable prosthesis with a RAR antagonist composition and implanting the coated prosthesis into the bone site, whereby such implantation promotes the formation of new bone formation.

According to a further embodiment of the invention, there is provided a method of producing bone at a bone defect site in vivo, the method comprising:

implanting into the defect site a population of osteoblastic cells or osteoblast progenitors which have been cultured in vitro in the presence of a RAR antagonist.

According to another embodiment of the invention, there is provided a method for treating a degenerative joint disease characterized by bone degeneration, the method comprising:

delivering a therapeutically effective amount of a RAR antagonist to a disease site.

The present invention in another aspect provides therapeutic compositions and methods for the treatment of disorders involving undesirable osteogenesis, ie. increased undesirable bone formation as is the case in ectopic bone formation and in osteopetrosis and fibrodysplasia ossificans progressiva (FOP) for example. Such pharmaceutical compositions comprise a therapeutically effective amount of an RAR angonist and a pharmaceutically acceptable carrier therefor.

In a further aspect of the invention, the invention embodies a pharmaceutical composition for the treatment of undesirable osteogenesis, for treatment of diseased bone tissue, or for inhibiting natural bone formation wherein the composition comprises a therapeutically effective amount of a RAR agonist and a pharmaceutically acceptable carrier. Such compositions may be used in methods for decreasing bone tissue in a mammal in need of such treatment. The method comprising administering a therapeutically effective amount of a RAR agonist to a mammal, wherein said RAR agonist inhibits bone mineralization and stimulates apoptosis of cells involved in bone tissue and bone tissue formation. The RAR agonist may be formulated to be targeted to a particular bone site.

In still another aspect of the invention, the invention provides a method for ex vivo skeletal tissue engineering, the method comprises culturing a population of cells in the presence of a RAR antagonist composition; and applying said cells to an implantable matrix and further incubating for a time sufficient for the cells to undergo osteogenesis; wherein the implantable matrix has bone tissue formation incorporated thereon and therein.

### BRIEF DESCRIPTION OF THE DRAWINGS

The present invention will be further understood from the following description with reference to the Figures, in which:

FIGS. 1A through F illustrate the inhibition of mineralization in MC3T3-E1 cultures with 1,25 VD₃ or an RAR-selective agonist. FIGS. 1A-D are photomicrographs in which cultures were treated with various concentrations of 1,25 VD₃ or AGN193836 for 28 days, fixed and stained with alizarin-red S. FIG. 1A, untreated culture; FIG. 1B, 1000 nM AGN193836; FIG. 1C, 10 nM 1,25 VD₃; FIG. 1D, 1000 nM AGN193836 and 10 nM 1,25 VD₃. FIG. 1E shows a graph illustrating the quantification of the amount of Alizarrin Red S stained matrix in MC3T3-E1 cultures after 28 days. The area occupied by Alizarrin Red S is expressed as the percentage of the total area that is Alizarrin Red S stained. Increasing concentrations of either agonist leads to a concentration-dependent decrease in area of calcified matrix. The asterisk denotes that the area occupied by Alizarin Red S stained material represents <0.1% of the total area. FIG. 1F shows a graph illustrating that 1,25 VD₃ and RAR-selective agonists reduce cell viability. Treatment of MC3T3-E1 cultures for 60 hr with various concentrations of either agent alone or in combination lead to a decrease in cell viability as measured by a decrease in absorbance at 595 nm with the MTT assay. Scale bar represents 0.5 mm.

FIGS. 2A through N illustrate the stimulation of apoptosis in MC3T3-E1 cultures treated with 1,25 VD₃ or RAR-selective agonist. FIGS. 2A-D are photomicrographs in which MC3T3-E1 cultures were treated with 1000 nM AGN193836, FIG. 2B, 10 nM 1,25 VD₃, FIG. 2C or both FIG. 2D for 72 hr and stained with PI. FIG. 2E is a graph showing the number of PI-stained cells (per unit area of 3 mm²) is increased with 1,25 VD₃ or AGN193836, and is further increased in the presence of both compounds. FIGS. 2F-N are photomicrographs showing an analysis of apoptosis in treated cultures with TUNEL. FIGS. 2F, 2G, 2H, untreated cultures; FIGS. 2I-N, cultures supplemented with 1000 nM AGN193836 and 10 nM 1,25 VD₃. Cultures were stained with PI (F, I, L) or TUNEL (G, J, M) and composite images were generated (H, K, N). Scale bar represents 0.3 mm for A-D, 0.08 mm for F-K and 0.65 mm for L-N.

FIGS. 3A through F illustrates the addition of a RARα-selective antagonist stimulates mineralization and reverses the effects of 1,25 VD₃. FIGS. 3A, B, C, D, cultures were treated with no ligand, 1000 nM AGN194301, 10 nM 1,25 VD₃ or both, respectively. FIG. 3E, is a graph showing the quantification of mineralization of 28 day old-MC3T3-E1 cultures. The extent of mineralization is expressed as a percentage of the total area occupied by alizarin-red S-stained material. Scale bar represents 0.5 mm. FIG. 3F shows a Northern blot in which MC3T3-E1 cultures were maintained under mineralizing conditions and the amount of OC mRNA was measured at various times after culture initiation using Northern blotting (upper panel) in treated and 1000 nM AGN194301-treated cultures. Lower panel, ethidium bromide stained gel showing abundance of 28S rRNA.

FIGS. 4A through E shows that an RAR-selective antagonist decreases cell death in 1,25 VD₃-treated cultures. MC3T3-E1 cells were treated for 36 hr with no ligand (FIG. 4A), 1000 nM AGN194301 (FIG. 4B), 10 nM 1,25 VD₃ (FIG. 4C) or both (FIG. 4D). FIG. 4E is a graph showing the number of PI-stained cells were counted per unit area in cultures treated with the indicated ligands. Scale bar represents 0.36 mm.

FIGS. 5A through 5 shows dominant-negative RAR-EGFP and RXR-EGFP fusion proteins localize to the nucleus and inhibit RA-mediated signaling. FIG. 5A is a graph measuring luciferase activity of COS cells transfected with the indicated dominant-negative constructs and their effect on RA-signaling was measured by co-transfection with an RARE-containing reporter in the presence or absence of RA. FIGS. 5B,C, D, E are images in which COS cells were transfected with either a dnRAR-EGFP (FIGS. 5B,C) or dnRXR-EGFP (FIGS. 5D,E) to determine the intracellular localization of the fusion proteins. FIGS. 5B, D, are phase contrast images of transfected cells, FIGS. 5C, E, correspond to epifluorescence images showing nuclear localization of EGFP. Scale bar represents 0.1 mm.

FIGS. 6A through K show the expression of a dnRAR or dnRXR in MC3T3-E1 cells inhibits 1,25 VD₃-mediated cell death. Cells were transfected with pSG5-EGFP (FIGS. 6A-D), pSG5-dnRARα-EGFP (FIGS. 6E-H) or dnRXRα-EGFP(FIGS. 6I, J) and treated with no ligand (FIGS. 6A, C, E, G, I) or 10 nM 1,25 VD₃ and 1000 nM AGN193836 (FIGS. 6B, D, F, H, J) for three days (FIGS. 6A-D, G-J) or four days (FIGS. 6E, F). FIG. 6K shows the number of EGFP-expressing cells that were counted in each treatment. Cultures in FIGS. 6A-F, I and J were stained with PI. Scale bar represents 0.4 mm for FIGS. 6A-B and 0.1 mm for FIGS. 6C-J.

FIGS. 7A through L show the effect of all-trans RA and AGN194301 on cell death and bone mineralization in normal human osteoblasts. FIGS. 7A, 7D and 7G represent untreated control cultures. FIGS. 7B, 7E and 7H are cultures treated with 1000 nM AGN194301. FIGS. 7C, 7F and 7I are cultures exposed to 1000 nM all-trans RA. Cells were cultured for 10 days and stained with either Hoechst 33342 (A-C) or PI (D-F). Fifteen day old cultures were stained with alizarin red (G-I). Treatment with all-trans RA leads to increased cell death, decreased cell number and reduced alizarin red staining in comparison to control cultures. FIGS. 7J and 7L, normal human osteoblasts were stained after 19 days of ligand treatment for calcium phosphate (black stained areas) using the Von Kossa method. FIG. 7J, untreated cultures. FIGS. 7K, 7L treated with 1000 nM AGN194301. Treatment of normal human osteoblast cultures with AGN194301 stimulates mineral deposition in comparison to control cultures. Magnification, bar equals 0.4 mm in 7A-F, 0.8 mm in 7G-K and 0.2 mm in 7L.

In the drawings, preferred embodiments of the invention are illustrated by way of example. It is to be expressly understood that the description and drawings are for the purpose of illustration and as an aid to understanding, and are not intended as a definition of the limits of the invention.

### DETAILED DESCRIPTION OF THE PREFERRED EMBODIMENTS

The present invention provides compositions and methods of use for affecting osteogenesis, either by stimulation or by inhibition of RAR. As such the invention has widespread clinical use to treat metabolic or non-metabolic bone diseases as well as diseases involving increased in appropriate bone formation.

The compositions of the invention have use both in vitro and in vivo as well as in ex vivo bone tissue engineering applications. The compositions also have particular use together with other physiological agents and in conjunction with prosthetic devices surgical implants of either resorbable or non-resorbable nature.

The present invention demonstrates that VDR and RAR-mediated signaling pathways cooperate to regulate preosteoblastic cell fate and differentiation. While VDR is thought to function predominantly through a VDR/RXR heterodimer, RAR-mediated signaling through direct or indirect mechanisms may be important in regulating VDR transcriptional activity, promoter specificity and/or cofactor recruitment. VDR and RARs are abundantly expressed in osteoblasts and their ligands have similar phenotypic effects, further suggesting an overlapping role in control of osteoblast function.

1,25 VD₃ has a well established function in maintaining calcium homeostasis. In this manner, 1,25 VD₃ concentrations influence osteoblast physiology indirectly through stimulation of resorption and elevation of Ca²⁺ (1). For this reason, it has been difficult to distinguish between the direct and indirect effects of 1,25 VD₃ on osteoblast function. Using a characterized osteoblastic cell line, MC3T3-E1, it is now demonstrated that under the appropriate conditions these cells can be treated to mineralize. During this process of differentiation, these cells pass through a number of well-defined stages which parallel those observed in vivo (20,24). Addition of 1,25 VD₃ to MC3T3-E1 cell cultures (or fetal calvaria cells of mouse or rat origin) early after culturing leads to a reduction in osteogenic nodule formation and decreased expression of osteocalcin (26-28).

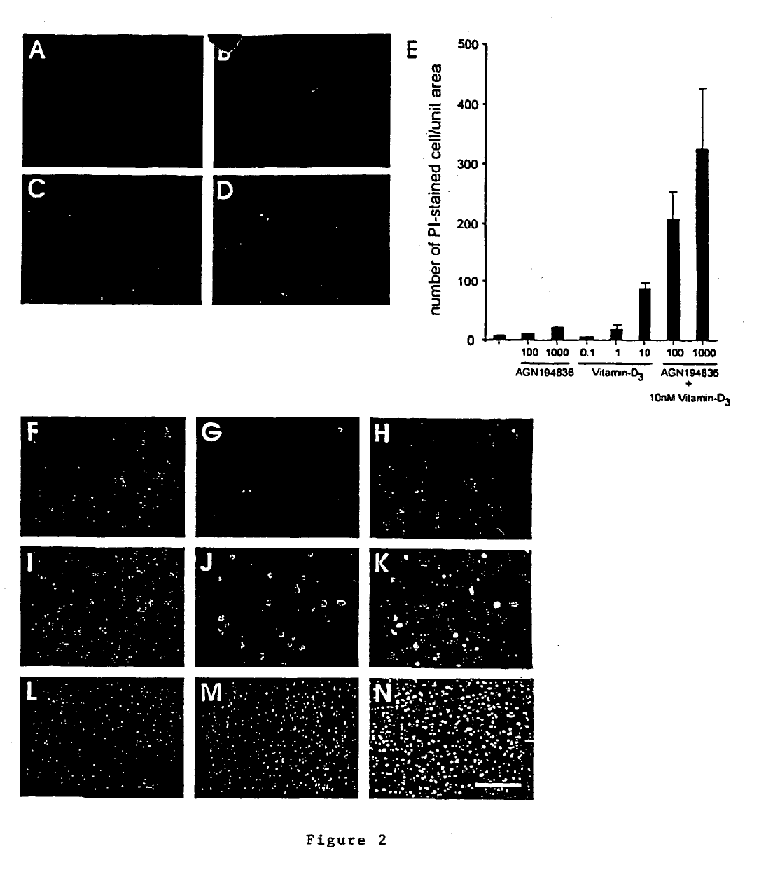

Consistent with these earlier studies, it is presently demonstrated that addition of 1,25 VD₃ to early stage cultures leads to a concentration-dependent decrease in mineralization. Under these conditions, however, it is also now first demonstrated that 1,25 VD₃ decreased cell viability with a concomitant increase in the appearance of apoptotic cell bodies. This suggests that part of the inhibitory action of 1,25 VD₃ on pre-osteoblasts resides with the ability of 1,25 VD₃ to stimulate apoptosis in these cell populations. Similarly, treatment of these same cultures with all-trans RA, 9-cis RA, TTNPB, and RARα-selective agonist (FIG. 2, and data not shown) inhibited mineralization and led to a concentration-dependent decrease in cell viability coupled with an increase in the number of dying cells. Hence, 1,25 VD₃ and retinoids elicited similar phenotypic responses in MC3T3-E1 cultures.

To assess potential cooperation between the two signaling pathways, the ability of the ligands to inhibit osteogenesis and stimulate cell death in combination was investigated. Combinations of ligands were found to be more effective than either alone and, in some cases, especially in induction of cell death, the two ligands appeared to be operating in a synergistic manner. Of particular interest is that the RAR-selective ligands operated similarly to all-trans or 9-cis, which can activate RXRs at high and low concentrations, respectively. In some instances, activation of the RXR moiety in this complex can inhibit 1,25 VD₃ mediated transactivation (29). However, the observation that RAR-selective ligands ellicited similar responses to ligands capable of activating either RARs or RXRs suggests an important requirement for activation of RAR-mediated signaling. Further evidence for the importance of RAR-signaling in this process is that the effects of 1,25 VD₃ on preosteoblastic cell death, and to a lesser extent mineralization, can be inhibited through the addition of an RARα-selective antagonist. Concentrations of the antagonist which are RARα-selective enhanced mineralization 3-fold in comparison to untreated cultures. Together, these data suggest a dependence on RAR-mediated signaling, and more specifically an important role for RAR in these processes.

To exclude receptor-independent mechanisms for the observed phenotypic changes, a dominant-negative version of RARα was constructed and tested for its ability to inhibit the action of 1,25 VD₃; a dominant-negative version of a known VDR interacting protein, RXR, was also made for comparative purposes. In the absence of selection, an increase in the number of EGFP-expressing cells in treated cells expressing either of the dominant-negatives as compared to cells transfected with EGFP alone was observed. The increase in the number of EGFP-expressing cells in the treated groups suggests that the presence of the dominant-negative receptor affords the cells expressing it with a selective advantage in the presence of 1,25 VD₃, an RAR-agonist or both. DnRARs and dnRXRs were both capable of inhibiting 1,25 VD₃-induced apoptosis. The dnRAR was slightly more potent in this respect, this is consistent with their respective effectiveness in inhibiting an RARE-reporter gene. In this context, it is possible that the dnRAR functions to sequester RXRs (30), and thereby limit VDR signaling indirectly through modulating accessibility of RXRs, an important heterodimeric VDR binding partner. However, the observation that RAR-selective antagonists which should not affect VDR/RXR signaling also inhibit 1,25 VD₃-induced preosteoblastic apoptosis further indicates a direct involvement of RARs.

Ligand-activated RARs have been shown to interfere with activating protein-1 (AP-1) activity and in some cell systems this leads to growth arrest and apoptosis (31). In combination, 1,25 VD₃ and retinoids have been shown to stimulate apoptosis (32). However, in most instances the ligands are not RAR-selective and in other cases the ligands are thought to have receptor-independent actions (32). Several RAR-selective antagonists exhibit anti-AP-1 which results in inhibition of cellular proliferation in a manner similar to that observed for retinoids (33). It is possible that AGN193836 and 1,25 VD₃ together could arrest growth and stimulate apoptosis through transrepression of AP-1. Addition of AGN194301 (if it does not operate to inhibit AP-1) may compete against endogenous RA and thereby reduce formation of ligand-activated RARs and reverse the effects of retinoid-activated receptors on AP-1. Both 1,25 VD₃ and retinoid-signaling pathways have been shown to cooperatively inhibit AP-1 activity, such that inactivation of one pathway might be sufficient to restore adequate AP-1 activity to maintain viability (34). However, evidence to suggest that inhibition of AP-1 is not enough to adequately explain the action of these two signaling pathways comes from the expression of dnRARs. In a previous study, Schule et al. (35) demonstrated that a dnRAR was ineffective at suppressing RA-mediated inactivation of AP-1 activity. However, as shown herein a dnRAR was effective at inhibiting the action of 1,25 VD₃, retinoids or both in induction of apoptosis in osteoblasts. These results do suggest that the action of the two pathways cannot be entirely explained by ligand-activated receptor transrepression of AP-1.

It is possible that VDR and RAR may associate in vivo to form heterodimers or larger heteromeric complexes to affect gene expression. VDR and RAR have been shown to cooperatively bind certain hormone response elements in vitro (11). However, no significant interaction between these two proteins using a mammalian two-hybrid system has been thus detected (Sampaio and Underhill, unpublished data). Similarly, recent studies have shown that VDR and TR also do not interact in vivo, while earlier studies performed in vitro suggested their possible interaction (36). Therefore, the mechanism by which VDR and RAR may interact to affect gene expression is still unknown.

The results of the present invention suggest that VDR and RAR-mediated signals converge to regulate programmed cell death in preosteoblasts. This in turn, suggests that expression of the osteoblastic phenotype is coordinated by a combination of VDR and RAR-mediated signals. In vivo, chronic exposure of dialysis patients to 1,25 VD₃ contributes to development of adynamic bone disease, which is characterized by significantly reduced bone formation and similar in outcome to that observed herein with chronic 1,25 VD₃ treatment of osteoblastic cultures (38). Simultaneous activation of both pathways in bone-forming cells also leads to a greater decrease in mineralization and under certain circumstances may be contributing factors to the development of skeletal diseases such as osteoporosis (16,17,39). Many of these effects of 1,25 VD₃ on preosteoblasts can be attenuated by interfering with RAR-mediated signaling, thus providing a potential therapeutic target with which to augment bone formation for the treatment of osteogenic disorders.

1,25 VD₃ and Retinoids Inhibit Mineralization of MC3T3-E1 Cultures

1,25 VD₃ and retinoids have been shown to influence skeletogenesis both in vitro and in vivo. Most of the effects of 1,25 VD₃ are thought to be mediated by the action of VDR, acting either as a homodimer or with an RXR heterodimeric partner. Retinoids can modulate the activity of this later complex or function through an RAR/RXR heterodimer to affect gene expression. To specifically address the contribution of RARs and VDRs to osteogenesis, RAR-selective agonists were used to activate RAR-mediated signaling.

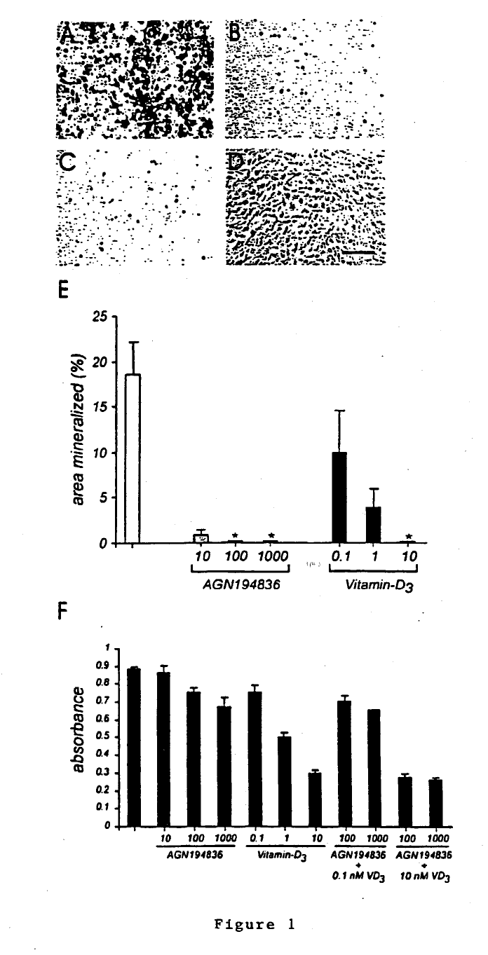

The effects of RAR-selective agonists on osteogenesis were studied in a well-characterized preosteoblastic cell line, MC3T3-E1, which has been found to closely follow the in vivo osteogenic program (20,24). Cells were allowed to reach confluence, at which time, ascorbate and GP were added along with ligands. After 28 days, cultures were fixed and the extent of mineralization determined by staining with Alizarin Red S. Approximately 18% of the field in control cultures was stained with Alizarin Red S (FIGS. 1A, E). In contrast, exposure of cultures to either an RAR-selective agonist (AGN193836) (25), 1,25 VD₃ or both lead to decreased in Alizarin Red staining in a dose-dependent manner (FIGS. 1B-E). Treatment of the cultures with AGN193836 at 10 nM dramatically inhibited bone formation, while higher concentrations completely abrogated bone formation (FIGS. 1B, E). Treatment with various concentrations of all-trans RA, 9-cis RA or TTNPB led to a similar decrease in bone formation (data not shown). Similarly, 1,25 VD₃ (0.1 nM) led to a decrease in the amount of Alizarin Red-staining, with increasing concentrations further suppressing bone formation (FIGS. 1C, E). There was a further reduction in bone nodule formation in combination, than with either treatment alone (FIGS. 1B, C, D). Interestingly, there was also a change in cellular morphology at higher drug concentrations, and this was especially evident in the cultures treated with both compounds (FIG. 1D). In these cultures, cells were stellate in appearance and the cell density was lower with individual cells being easily discerned (compare FIG. 1A with 1D). In treated cultures, the cells remained as a single layer, whereas in the untreated cultures the cells form multiple layers. Consistent with the apparent changes in cell density in long-term cultures, analysis of cell viability using an MTT assay after 60 hr showed that treatment of MC3T3-E1 cells with AGN193836 or 1,25 VD₃ individually or in combination decreased cell viability (FIG. 1F). In summary, treatment of preosteoblasts with 1,25 VD₃ or an RAR-selective agonist leads to similar inhibition of mineralization and cell viability and, effects which are more pronounced in combination.

1,25 VD₃ and Retinoids Stimulate Apoptosis of Preosteoblasts

Treatment of differentiating osteoblasts with both 1,25 VD₃ and retinoids led to a marked decrease in cell density. This was accompanied at early culture times (2-3 days after drug addition) with the appearance of numerous detached cells in the medium. To assess the viability of these floating cells, a membrane-impermanent nuclear stain, propidium iodide, was added to the medium. The floating bodies became fluorescent in the presence of PI, suggesting that their membranes were compromised and that they represented dead or dying cells (FIGS. 2B-E). As was observed in the mineralizing cultures, increasing concentrations of either 1,25 VD₃ or AGN193836 led to increased numbers of PI-positive cells (FIGS. 2B-E). A modest increase of 2-3 fold in the number of fluorescing cells was observed with increasing concentrations of AGN93836. All-trans RA, 9-cis RA and TTNPB were also found to stimulate cell death (data not shown). Similarly, increasing concentrations of 1,25 VD₃ led to a greater number of PI-stained bodies (FIGS. 2C, E), with 1,25 VD₃ exhibiting a greater potency than the RAR-agonist over the concentration range examined. However, in combination, there was a dramatic increase in the number of PI-stained cells that was greater than the sum of the effects of the two ligand treatments individually, indicating synergism (FIGS. 2D, E). Hence, it appeared that 1,25 VD₃ and retinoids stimulated cell death in differentiating osteoblasts. The morphology and intense PI-staining of these bodies was consistent with condensed chromatin in apoptotic bodies.

To further address whether the ligands caused cell death through programmed cell death, TUNEL labeling was used to detect apoptotic cells. Cells were fixed and labeled using the TUNEL assay, followed by staining with propidium iodide. Under these conditions all of the cells are PI-positive due to fixation prior to staining, however, there appears to be two predominant cell populations, one with weak diffuse PI staining, and the other with much more intense PI staining, reflective of chromatin condensation. Cells staining most intensely for PI were similar in morphology to those observed in earlier experiments, and they were found to be very abundant in cultures treated with both ligands (FIGS. 2I, L), as compared to control cultures (FIG. 2F). It was also this group of cells which stained positively with TUNEL (FIGS. 2G, H, J, K, M, N). As was observed with PI staining, combined treatment with both ligands greatly increased the number of TUNEL-positive cells (compare FIGS. 2G to 2J, M). TUNEL-positive cells were also observed following treatment with individual ligands, albeit fewer in comparison to treatment with both ligands (data not shown). Furthermore, it was found that addition of the ligands to cultures at low density (<60% confluence) slowed the appearance of apoptotic cells, in comparison to treatment of high-density cultures (>95% confluence) where apoptotic cells were observed after 24 hrs (data not shown). Induction of apoptosis appeared to be dependent upon the degree of cell density. These results suggest that retinoids and 1,25VD₃ stimulate apoptosis in differentiating osteoblastic cultures, and it is likely this propensity which contributes to inhibition of bone nodule formation. Analysis of these results suggests that VDR and RAR-mediated signals function cooperatively to inhibit mineralization and to stimulate apoptosis in osteoblast cultures.

An RAR-selective Antagonist Stimulates Mineralization and OC Expression

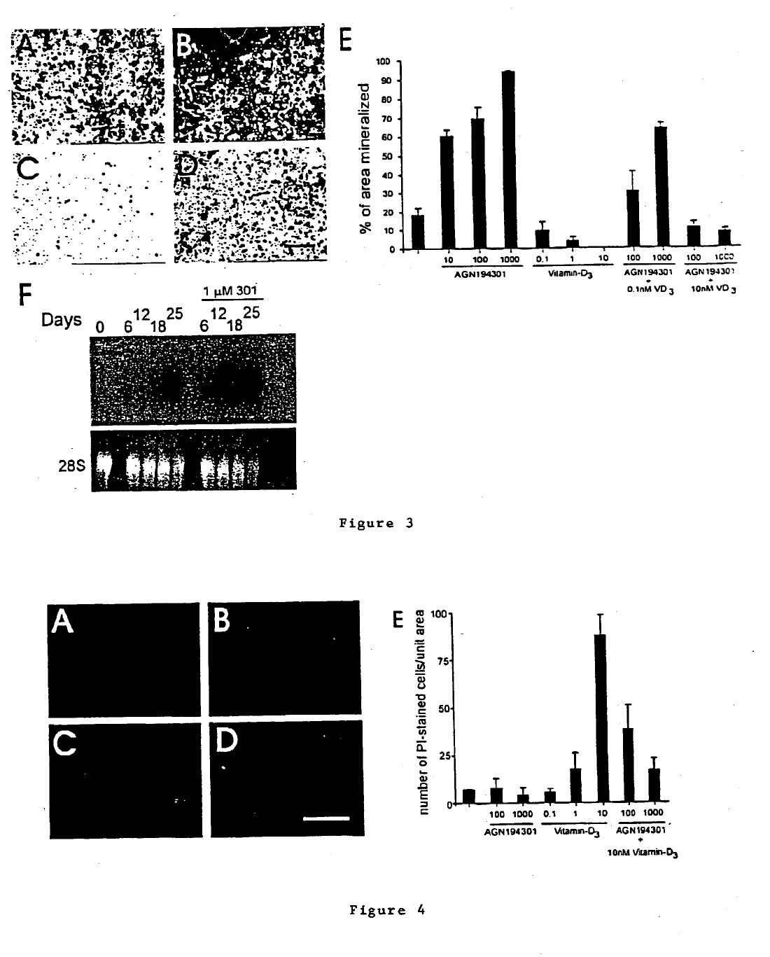

To further address the nature of the cooperation between VDR and RAR signaling pathways, an RAR-selective antagonist was used to evaluate its ability to inhibit the action of 1,25 VD₃. Treatment of mineralizing MC3T3-E1 cultures with the RARα-selective antagonist, AGN194301 (27), led to a concentration-dependent increase in Alizarin Red S staining (FIGS. 3A, B, E). Concentrations as low as 10 nM AGN194301 increased Alizarin Red S staining by an amount approximately 3 fold greater than untreated controls (FIG. 3E). At 1 μM approximately 95% of the culture surface area was stained with Alizarin Red S, in comparison to control cultures with approximately 18% staining (FIGS. 3A, B, E). In accordance with these observations, addition of 1000 nM AGN194301 to MC3T3-E1 cultures stimulated OC expression (FIG. 3F). The antagonist increased steady-state levels of OC mRNA in 25 day-old cultures in comparison to control cultures, and also accelerated the appearance of osteocalcin mRNA in these cultures. In wild-type cultures, OC mRNA first became apparent after 18 days of culture, whereas in the antagonist-treated cultures strong OC mRNA expression became evident after 12 days (FIG. 3F). This increase in OC mRNA abundance in antagonist-treated cultures is consistent with its effects on bone mineralization.

The results with the RARα antagonist are in marked contrast to those observed with an RAR-selective agonist (FIG. 1B). Moreover, addition of the antagonist to 1,25 VD₃-treated cultures increased the amount of bone nodule formation. At 10 nM 1,25 VD₃, addition of 100 nM AGN194301 restored bone formation to ˜70% of that in control cultures (FIGS. 3A, C-E), however, this was much lower than that observed in the antagonist-alone treated cultures. Thus, addition of RAR-selective antagonist could partially reverse the effects of 1,25 VD₃ on mineralization. Consistent with these results, addition of AGN194301 to 1,25 VD₃-treated cultures also inhibited 1,25 VD₃-induced cell death (FIGS. 4A-E). AGN194301 alone had no appreciable effect on the appearance of PI-positive cells in MC3T3-E1 cultures (FIGS. 4A, B). However, addition of the antagonist dramatically reduced the number of PI-positive cells in 1,25 VD₃-treated cultures to an amount less than 20% of that of 1,25 VD₃ treated culture (FIGS. 4C-E). Thus, an RAR-selective antagonist is able to reverse the effects of 1,25 VD₃ on apoptosis and in part, on mineralization.

Expression of a Dominant Negative RAR or RXR Inhibits 1,25 VD₃-mediated Apoptosis

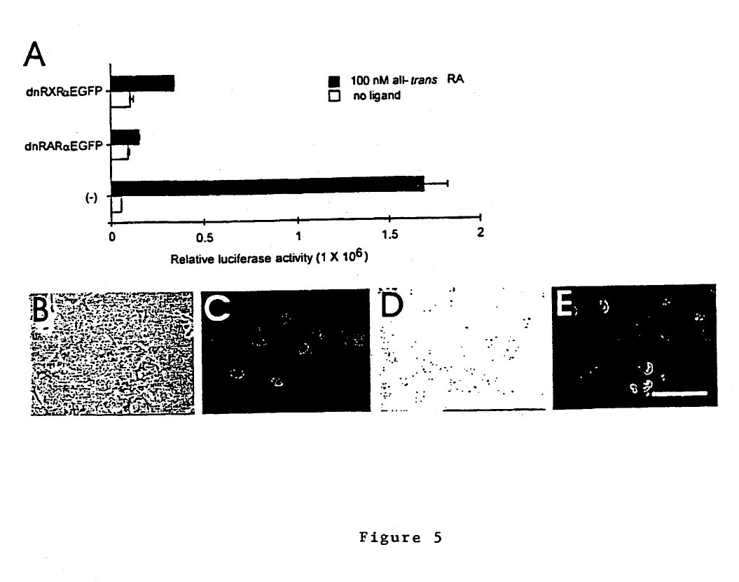

To determine if 1,25 VD₃ was operating through an RAR-mediated pathway, dominant-negative versions of RARα and a well-defined partner of VDR, an RXR, were constructed. To monitor cells expressing the dominant-negative receptors, the constructs incorporated a C-terminal fusion to enhanced green fluorescent protein (EGFP). In this respect, the fate of individual cells in the presence or absence of ligands could be followed. More importantly, this eliminated the need to establish stable clones and minimized the problems inherent with clonal heterogeneity. DnRAR-EGFP and dnRXR-EGFP were initially tested in COS P7 cells and found to inhibit RA stimulation of an RARE containing reporter gene, and to localize to the nucleus (FIGS. 5A-E).

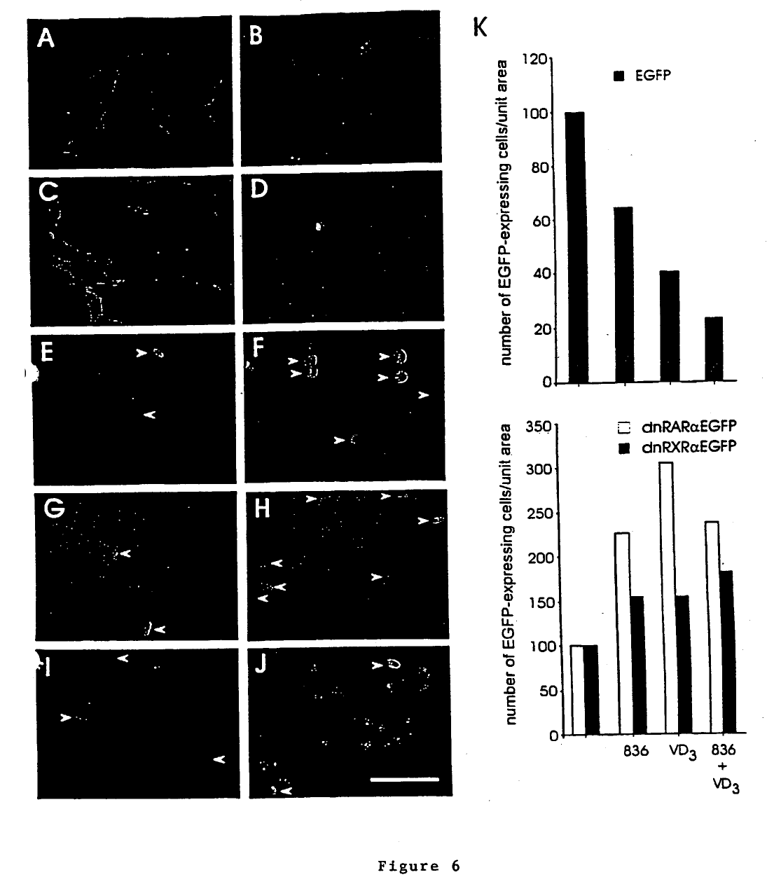

The dn receptor constructs were transfected into MC3T3-E1 cells, followed by treatment of the cells with 1000 nM RAR-agonist and/or 10 nM 1,25 VD₃. In control cells expressing EGFP alone, there was a decrease in the number of fluorescing cells present in the individual treatments, with the greatest decrease being observed in the co-treated cultures (FIGS. 6A-D, K). The decline in EGFP-expressing cells is consistent with the decrease in cell viability and increase in cell death in ligand-treated cultures described above (FIG. 1F and FIG. 2E). In contrast, the number of cells expressing the dnRAR-EGFP increased in cultures treated with the ligands alone or in combination, with a 3-fold increase in the number of fluorescing cells per unit area in the cultures treated with 1,25 VD₃ alone as compared to untreated controls (FIGS. 6E-H, K). Expression of a dnRXR-EGFP also protected MC3T3-E1 cells from 1,25 VD₃-induced cell death, albeit less effectively than that observed for dnRAR-EGFP. As was found in the COS-transfected cells, the dnRAR-EGFP and dnRXR-EGFP localized to the nucleus in MC3T3-E1 cells. Thus, inhibition of RAR-mediated signaling either through the addition of an RAR-selective antagonist or transfection of a dnRAR is sufficient to inhibit the action of 1,25 VD₃ on osteoblastic cells.

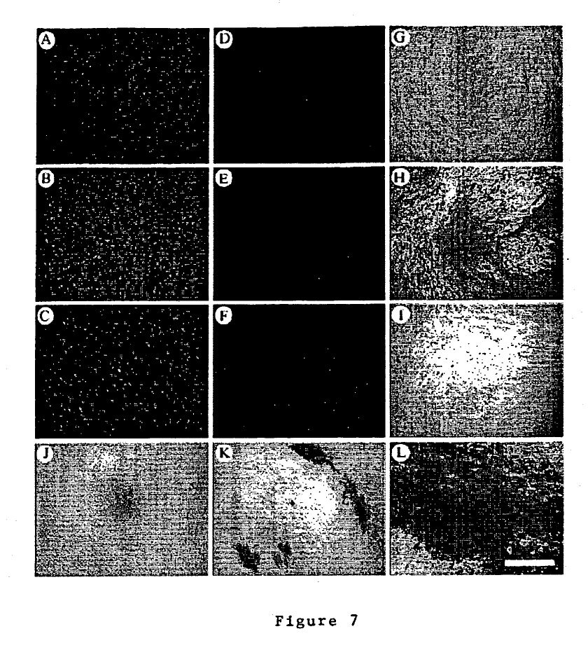

The effects of RA and RAR antagonists were also demonstrated in normal human osteoblast cultures (NHO). Cells were stained with a DNA stain to allow visualization of the cell nuclei and allow assessment of cell number (FIGS. 7A-C). FIGS. 7D-F the cells were stained with a membrane impermeant nucleic acid stain to allow visualization of dead or dying cells. Treatment with all-trans RA appeared to increase the number of PI-stained cells suggesting that all-trans RA stimulates cell death in these cultures. In FIGS. 7G, 7I the cells were stained with alizarin red S. The antagonist treated cultures exhibit more alizarin red S staining suggesting that the antagonist promotes bone formation in cultures of normal human osteoblasts. In FIGS. 7J-7L the cultures were stained to examine phosphate deposits. The cultures treated with the antagonist exhibit enhanced phosphate staining, indicating that the antagonist promotes bone nodule formation of cultures of normal human osteoblasts. Collectively, this human in vitro data demonstrates the effectiveness of RAR antagonists to stimulate bone formation and RA to stimulate apoptosis in these cells.

The RAR antagonist AGN 194301 has been demonstrated to decrease 1,25 VD₃ induced apoptotis in osteoblasts and also to stimulate and promote osteoblast differentiation and mineralization. AGN 194301 (2-Fluoro-4-[(1-(8-bromo-2,2-dimethyl-4-(4-methylphenyl)-2-H-chromen-6-yl)-methanoyl)-amino]-benzoic acid) is a potent antagonist of RARα, with a high affinity for that receptor. It has a lower affinity for RARβ and RARγ, but does also act as an antagonist of these receptors.

In accordance with one embodiment of the invention, osteogenesis-stimulating RAR antagonists comprise antagonist compounds which are highly effective against RARα and also antagonise RARβ and RARγ. Thus, the present invention encompasses RAR antagonists in general, analogues thereof, and any agent which demonstrates RAR antagonist activity. Those of ordinary skill in the art are able to screen candidate compounds to identify compounds having such an RAR antagonist profile by methods available in the scientific literature, for example as described in Teng et al., (1997), J. Med. Chem., 40, 2445-2451. Therefore, one skilled in the art would understand that the invention is not limited to those RAR antagonists as used and specifically described herein, but would contemplate that any agent demonstrated to have RAR antagonist activity would be successfully encompassed in the present invention. Furthermore, one skilled in the art would understand that mixtures of RAR antagonists would also be encompassed in the compositions of the present invention.

In accordance with one embodiment of the invention, osteogenesis-stimulating RAR antagonists comprise mono- or di-fluoro substituted methylchromenes such as AGN 194301. The RAR antagonist compounds of the invention may be synthesized by conventional chemical synthetic methods. For example, AGN 194301 may be synthesized as described in Teng et al., (supra) or as described in U.S. Pat. No. 5,559,248, which is incorporated herein by reference in its entirety. Other useful RAR antagonists are described in, for example, Eyrolles et al., Med. Chem. Res. 2:361-367 (1992) and Apfel et al., Proc. Natl. Acad. Sci. USA 89:7129-7133 (1992), which are incorporated by reference herein in their entireties.

Again, one skilled in the art would readily understand that several different types of RAR antagonists other than those described specifically herein are suitable for use in the present invention. Other suitable RAR antagonists are taught for example in WO 9933821, WO 9924415, U.S. Pat. No. 5,877,207, U.S. Pat. No. 5,776,699 and JP 10114757 (the disclosures each of which are in entirety herein incorporated by reference). Such antagonist agents include but are not limited to AGN 193109, AGN 190121, AGN 194574, AGN 193174, AGN 193639, AGN 193676, AGN 193644, SRI 11335, Ro 41-5253, Ro 40-6055, CD 2366, BMS 185411, BMS 189453, CD-2665, CD 2019, CD 2781, CD 2665, CD 271. Other suitable RAR antagonists for use in the present invention include those disclosed in Kaneko et al., 1991; Eyrolles et al., 1994; Yoshimura et al., 1995; Eckharat and Schmitt, 1994; and Teng et al., 1997. It is also within the scope of the present invention to use mixtures of RAR antagonists as desired.

With the demonstration that RAR antagonists can directly affect bone formation in vivo and in vitro and in particular possess osteogenesis-promoting activity, pharmaceutical compositions can now be developed and used in order to treat a host of bone development abnormalities (both non-metabolic bone diseases and metabolic bone diseases) or bone trauma as well as hypervitaminosis A and vitamin D toxicity. Representative uses of the RAR antagonists of the present invention for bone development abnormalities or bone trauma include for example repair of bone defects and deficiencies, such as those occurring in closed, open and non-union fractures, bone/spinal deformation, osteosarcoma, myeloma, bone dysplasia and scoliosis; prophylactic use in closed and open fracture reduction; promotion of bone healing in plastic surgery; stimulation of bone ingrowth into non-cemented prosthetic joints and dental implants; elevation of peak bone mass in pre-menopausal women; treatment of growth deficiencies; treatment of periodontal disease and defects, and other tooth repair processes; increase in bone formation during distraction osteogenesis; and treatment of other skeletal disorders, such as age-related osteoporosis, post-menopausal osteoporosis, glucocorticoid-induced osteoporosis or disuse osteoporosis and arthritis, osteomalcia, fibrous osteitis, renal bone dystrophy and Paget's disease of bone, or any condition that benefits from stimulation of bone formation.

One skilled in the art would be able to use RAR antagonist compositions as described herein to treat and alleviate the aforementioned bone diseases and have a reasonable expectation of success with respect to a positive physiological effect on a variety of cell types including but not limited to embryonic stem cells, adult stem cells, osteoblastic cells, preosteoblastic cells and skeletal progenitor cells derived from bone, bone marrow or blood. It is also encompassed within the present invention to use the RAR antagonist compositions on dedifferentiated cells. Dedifferentiated cells are post-mitotic cells that have reentered the cell cycle and may contribute to other cell types. One skilled in the art would realize that dedifferentiated cells such as taken from muscle for example, may be treated with RAR antagonist composition to redifferentiate to continue to an osteoblastic potential. Any multipotential cell types may be used and treated with the compositions of the invention to continue to osteogenesis. Further, any number of agents such as bone morphogenetic factors, anti-resorptive agents, osteogenic factors, cartilage-derived morphogenetic proteins, growth hormones and differentiating factors may be used together with the RAR antagonist compositions of the invention in order to aid in the promotion of osteogenesis.

The compositions of the present invention can be useful in repair of congenital, trauma-induced or surgical resection of bone (for instance, for cancer treatment), and in cosmetic surgery. Bone deficit or defect can be treated in vertebrate subjects by administering the RAR antagonist compounds of the invention which exhibit certain structural and functional characteristics. The compositions of the invention may be administered systemically or locally. For systemic use, the compounds herein are formulated for parenteral (e.g., intravenous, subcutaneous, intramuscular, intraperitoneal, intranasal or transdermal) or enteral (e.g., oral or rectal) delivery according to conventional methods. For example, the composition may be shaped into suppositories such as rectal preparations, and non-oral preparations for topical administration (e.g. intramuscular, subcutaneous, intra-articular injections, embedding preparation, soft ointments, etc.).

For oral administration the compositions may be in the form of a liquid preparation as it is, or may be filled in soft capsules or like to yield an oral preparation when it is obtained in a liquid form. When the composition of the present invention is in a solid dispersion, it can be packed in capsules or shaped into pellets, fine granules, granules or tablets to yield an oral preparation. As a solid dispersion, the composition may be shaped into solid forms such as spheres, rods, needles, pellets and films in the presence of additional additives as necessary as is understood by one skilled in the art.

Intravenous administration can be by a series of injections or by continuous infusion over an extended period. Administration by injection or other routes of discretely spaced administration can be performed at intervals ranging from weekly to once to three times daily. Alternatively, the compounds disclosed herein may be administered in a cyclical manner (administration of disclosed compound; followed by no administration; followed by administration of disclosed compound, and the like). Treatment will continue until the desired outcome is achieved.

The RAR antagonist compositions are administered in a therapeutically effective dose in accordance with the invention. A therapeutic concentration will be that concentration which effects reduction of the particular condition (such as vitamin A toxicity) or retards its expansion. It should be understood that when coadministering the antagonist compounds to block retinoid-induced toxicity, the antagonist compositions are used in a prophylactic manner to prevent onset of a particular condition.

A useful therapeutic or prophylactic concentration will vary from condition to condition and in certain instances may vary with the severity of the condition being treated and the patient's susceptibility to treatment. Accordingly, no single concentration may be uniformly useful, but will require modification depending on the particularities of the chronic or acute bone condition being treated. Such concentrations can be arrived at through routine experimentation as is known to those of skill in the art. However, it is anticipated that a composition containing between 0.01 and 1.0 milligrams of antagonist per ml of formulation may constitute a therapeutically effective concentration for topical application for example. If administered systemically, an amount between 0.01 and 5 mg per kg per day of body weight may provide a therapeutic result. In general, compositions may be administered at a dosage range of from about 0.001 mg/kg of body weight to about an upper limit of 300 mg/kg of body weight.

In general, pharmaceutical formulations will include a RAR antagonist of the present invention in combination with a pharmaceutically acceptable vehicle, such as saline, buffered saline, 5% dextrose in water, ethanol, borate-buffered saline containing trace metals or the like and mixtures thereof. Formulations may further include one or more excipients, preservatives, solubilizers, buffering agents, albumin to prevent protein loss on vial surfaces, lubricants, fillers, stabilizers, etc. Methods of formulation are well known in the art and are disclosed, for example, in Remington's Pharmaceutical Sciences, Gennaro, ed., Mack Publishing Co., Easton Pa., 1990, which is incorporated herein by reference.

The compositions of the present invention can be used concomitantly with other agents for treating bone diseases. Examples of drugs concomitantly used may include for example, calcium preparations (e.g. calcium carbonate), calcitonin preparations, sex hormones (e.g. estrogen, estradiol), prostaglandin A1, bisphosphonic acids, ipriflavones, fluorine compounds (e.g. sodium fluoride), vitamin K, bone morphogenetic proteins (BMPs), fibroblast growth factor (FGF), platelet-derived growth factor (PDGF), transforming growth factor (TGF-β), insulin-like growth factors 1 and 2 (IGF-1,2), parathyroid hormone (PTH), epidermal growth factor (EGF), leukemia inhibitory factor (LIP), osteogenin, and bone resorption repressors such as estrogens, calcitonin and biphosphonates. It is also contemplated that mixtures of such agents may also be used and formulated within the compositions of the present invention or used in conjuction with the compositions of the present invention.

Pharmaceutical compositions for use within the present invention can be in the form of sterile, non-pyrogenic liquid solutions or suspensions, coated capsules, creams, lotions, suppositories, lyophilized powders, transdermal patches or other forms known in the art. Local administration may be by injection at the site of injury or defect, or by insertion or attachment of a solid carrier at the site, or by direct, topical application of a viscous liquid, or the like. For local administration, the delivery vehicle preferably provides a matrix for the growing bone or cartilage, and may be a vehicle that can be absorbed by the subject without adverse effects.

Delivery of the antagonist compounds herein to wound sites may be enhanced by the use of controlled-release compositions, such as those described in WIPO publication WO 93/20859 (which is incorporated herein by reference in its entirety). Films of this type are particularly useful as coatings for both resorbable and non-resorbable prosthetic devices and surgical implants. The films may, for example, be wrapped around the outer surfaces of surgical screws, rods, pins, plates and the like. Implantable devices of this type are routinely used in orthopedic surgery. The films can also be used to coat bone filling materials, such as hydroxyapatite blocks, demineralized bone matrix plugs, collagen matrices and the like. In general, a film or device as described herein is applied to the bone at the fracture site. Application is generally by implantation into the bone or attachment to the surface using standard surgical procedures.

In addition to the copolymers and carriers noted above, the biodegradable films and matrices incorporating the antagonist compositions may include other active or inert components and mixtures thereof as discussed supra. Of particular interest are those agents that promote tissue growth or infiltration, such as growth factors. Exemplary growth factors for this purpose include epidermal growth factor (EGF), fibroblast growth factor (FGF), platelet-derived growth factor (PDGF), transforming growth factors (TGFs), parathyroid hormone (PTH), leukemia inhibitory factor (LIF), insulin-like growth factors (IGFs) and the like. Agents that promote bone growth, such as bone morphogenetic proteins (U.S. Pat. No. 4,761,471), osteogenin (Sampath et al. Proc. Natl. Acad Sci USA (1987) 84:7109-13) and NaF (Tencer et al. J. Biomed. Mat. Res. (1989) 23: 571-89) are also preferred. Biodegradable films or matrices include calcium sulfate, tricalcium phosphate, hydroxyapatite, polylactic acid, polyanhydrides, bone or dermal collagen, pure proteins, extracellular matrix components and the like and combinations thereof. Such biodegradable materials may be used in combination with non-biodegradable materials (for example polymer implants, titanium implants), to provide desired mechanical, cosmetic or tissue or matrix interface properties.

Alternative methods for delivery of compounds of the present invention include use of ALZET osmotic minipumps (Alza Corp., Palo Alto, Calif.); sustained release matrix materials such as those disclosed in Wang et al. (PCT Publication WO 90/11366); electrically charged dextran beads, as disclosed in Bao et al. (PCT Publication WO 92/03125); collagen-based delivery systems, for example, as disclosed in Ksander et al. Ann. Surg. (1990) 211 (3):288-94; methylcellulose gel systems, as disclosed in Beck et al. J. Bone Min. Res. (1991) 6(11): 1257-65; alginate-based systems, as disclosed in Edelman et al. Biomaterials (1991) 12:619-26 and the like. Other methods well known in the art for sustained local delivery in bone include porous coated metal prostheses that can be impregnated and solid plastic rods with therapeutic compositions incorporated within them.

In one embodiment, the RAR antagonist composition may comprise at least one RAR antagonist which may be provided as a solution or emulsion contained within phospholipid vesicles called liposomes. The liposomes may be unilamellar or multilamellar and are formed of constituents selected from phosphatidylcholine, dipalmitoylphosphatidylcholine, cholesterol, phosphatidylethanolamine, phosphatidylserine, demyristoylphosphatidylcholine and combinations thereof. The multilamellar liposomes comprise multilamellar vesicles of similar composition to unilamellar vesicles, but are prepared so as to result in a plurality of compartments in which the silver component in solution or emulsion is entrapped. Additionally, other adjuvants and modifiers may be included in the liposomal formulation such as polyethyleneglycol, or other materials.

It is understood by those skilled in the art that any number of liposome bilayer compositions can be used in the composition of the present invention. Liposomes may be prepared by a variety of known methods such as those disclosed in U.S. Pat. No. 4,235,871 and in RRC, Liposomes: A Practical Approach. IRL Press, Oxford, 1990, pages 33-101.

The liposomes containing the RAR antagonist may have modifications such as having non-polymer molecules bound to the exterior of the liposome such as haptens, enzymes, antibodies or antibody fragments, cytokines and hormones and other small proteins, polypeptides or non-protein molecules which confer a desired enzymatic or surface recognition feature to the liposome. Surface molecules which preferentially target the liposome to specific organs or cell types include for example antibodies which target the liposomes to cells bearing specific antigens. Techniques for coupling such molecules are well known to those skilled in the art (see for example U.S. Pat. No. 4,762,915 the disclosure of which is incorporated herein by reference). Alternatively, or in conjunction, one skilled in the art would understand that any number of lipids bearing a positive or negative net charge may be used to alter the surface charge or surface charge density of the liposome membrane.

The liposomes can also incorporate thermal sensitive or pH sensitive lipids as a component of the lipid bilayer to provide controlled degradation of the lipid vesicle membrane.

For systemic application by intravenous delivery, it may be beneficial to encapsulate the RAR antagonist within sterically-stabilized liposomes which exhibit prolonged circulation time in blood. The sterically stabilized liposomes are produced containing polyethylene glycol as an essential component of their surface and the method of making such liposomes is known to those skilled in the art.

The size of the liposomes can be selected based on the intended target and route of administration. Liposomes of between about 10 nm to 300 nm may be suitable. Furthermore, the composition of the present invention may include liposomes of different sizes.

While the composition of the present invention may be encapsulated for administration by liposomes, it is understood by those skilled in the art that other types of encapsulants may also be used to encapsulate the RAR antagonist. Microspheres including but not limited to those composed of ion-exchange resins, crystalline ceramics, biocompatible glass, latex and dispersed particles are suitable for use in the present invention. Similarly, nanospheres and other lipid, polymer or protein materials can also be used.

The invention also provides compositions employing antisense based strategies in order to inhibit or reduce RAR gene function and thus RAR activity. The principle is based on the hypothesis that sequence specific suppression of gene expression can be achieved by intracellular hybridization between mRNA and a complementary anti-sense species. It is possible to synthesize anti-sense strand nucleotides that bind the sense strand of RNA or DNA with a high degree of specificity. The formation of a hybrid RNA duplex may then interfere with the processing/transport/translation and/or stability of a target mRNA.

Hybridization is required for an antisense effect to occur. Antisense effects have been described using a variety of approaches including the use of AS oligonucleotides, injection of AS RNA, DNA and transfection of AS RNA expression vectors. Therapeutic antisense nucleotides can be made as oligonucleotides or expressed nucleotides. Oligonucleotides are short single strands of DNA which are usually 15 to 20 nucleic acid bases long. Expressed nucleotides are made by an expression vector such as an adenoviral, retroviral or plasmid vector. The vector is administered to the cells in culture, or to a patient, whose cells then make the antisense nucleotide. Expression vectors can be designed to produce antisense RNA, which can vary in length from a few dozen bases to several thousand.

In the present invention, mammalian cells which express RAR can be additionally transfected with anti-sense RAR DNA sequences in order to inhibit the transcription of the RAR gene. Alternatively, the anti-sense RAR sequences can be administered as a composition. Suitable antisense oligonucleotides are directed to a portion of the RAR sequences which are deposited in GenBank.

In summary, RAR antagonists have important clinical therapeutic uses for treatment of bone development defects and bone toxicity. The RAR antagonists can be used to provide such treatment both in vitro, in vivo and ex vivo to treat a variety of conditions as a result of trauma, genetic disease or degenerative disease negatively affecting bone development and maintenance.

For in vitro and ex vivo tissue engineering use, one skilled in the art may apply a selected RAR antagonist or mixture thereof to a desired culture of cells. Representative cell cultures are described herein with reference to the examples but in general may include embryonic stem cells, adult stem cells, osteoblastic cells, preosterblastic cells and skeletal progenitor cells derived from bone, bone marrow or blood. Such cells may also include dedifferentiated cells. Cell cultures may be maintained until a desired physiological result is achieved after which the cells are administered by various conventional methods to patient at a desired tissue site. Alternatively, such cultured treated cells may be applied or growth within to an implant or within an implant or prosthetic device and further cultured in vitro to allow for bone mineralization and deposition to take place prior to patient implantation.

The present invention in a second embodiment provides RAR agonist pharmaceutical compositions for inhibiting osteogenesis for treating disorders where there is excessive bone formation as seen in ectopic bone formation and also for example in osteopetrosis or fibrodysplasia ossificans progressiva (FOP). RAR agonists comprise agonist compounds which initiate a cellular response when associated with a RAR.

Regardless of the mode of administration of the RAR agonist composition of the invention, the RAR agonist can be either naturally occurring or a synthetic retinoid, preferably having selective activity as an agonist for RARs. Examples of naturally occurring retinoids with activity as RAR agonists are all-trans retinoic acid (all-trans RA) and 9-cis retinoic acid (9-cis RA), which are stereoisomers, all-trans RA being naturally converted into 9-cis RA during metabolism (J. G. Allen, et al., Pharmac. Ther., 40:1-27, 1989).

Synthetically prepared retinoids are well known in the art. For example, U.S. Pat. No. 5,234,926, which is incorporated herein by reference in its entirety, discloses methods of synthesizing disubstituted acetylenes bearing heteroaromatic and heterobicyclic groups with selective activity as RAR agonists. U.S. Pat. No. 4,326,055, which is incorporated herein by reference in its entirety, discloses methods for synthesizing 5,6,7,8-tetrahydro naphthyl and indanyl stilbene derivatives with retinoid-like activity. Retinoid compounds can readily be selected by determining whether they have RAR activity, for instance by utilizing well known in vitro transacivation assay techniques such as that disclosed by M. Pfahl, et al., Methods in Enzymology, 1:256-270, 1990.

Examples of synthetic RAR agonists suitable for use in the practice of this invention are ethyl 6-[2-(4,4-dimethylthiochroman-6-yl)ethynyl]nicotinate and 6-[2-(4,4-dimethylchroman-6-yl)ethynyl]nicotinic acid whose synthesis is disclosed in U.S. Pat. No. 5,234,926; and p-[(E)-2-(5,6,7,8-tetrahydro-5,5,8,8-tetramethyl-2-naphthyl)propenyl]-benzoic acid whose synthesis is disclosed in U.S. Pat. No. 4,326,055. By contrast, an example of an RXR selective agonist is 2-[(E)-2-(5,6,7,8-tetrahydro-3,5,5,8,8-pentamethylnaphthaleen-2-yl)propen-1-yl]thiophene-4-carboxylic acid (Compound 701), whose synthesis is disclosed in U.S. Pat. No. 5,324,840.

Those of ordinary skill in the art are able to screen candidate compounds to identify compounds having a RAR agonist profile by methods known in the art. Further agonists for use in the compositions of the present invention may include but are not limited to any retinoid compound in general, TTMPB, AGN 193836 and LG1069, the structure and preparation of which are described in Boehm et al., J. Med. Chem. 37:2930-2941 (1994), which is incorporated by reference herein in its entirety. Other useful RAR agonists are described in, for example, Lehmann et al., Science 258:1944-1946 (1992), which is incorporated by reference herein in its entirety.

Other RAR agonists suitable for use in the present invention may be prepared by the above-cited methods and others routine to those of ordinary skill in the art and would be expected by one skilled in the art to have a reasonable expectation of physiological success for the inhibition of osteogenesis in a variety of cell types such as for example embryonic stem cells, adult stem cells, osteoblastic cells, preosteoblastic cells and skeletal progenitor cells derived from bone, bone marrow or blood. Such cells may also include dedifferentiated cells obtained from various tissues such as muscle for example.

As with the RAR antagonist compositions of the present invention, the RAR agonist compositions can be used in vitro, in vivo and in ex vivo tissue engineering and can be formulated and used in the various physiological and clinical applications as is previously described in the above text for RAR antagonist compositions.

It is also encompassed that the RAR antagonist and RAR agonist compositions of the present invention can be used in conjunction to treat various osteological conditions necessitating osteogenesis stimulation and osteogenesis inhibition at different time periods during treatment.

The osteogenesis promoting and inhibiting pharmaceutical compositions of the present invention and the preparation based thereon as well as the methods employing such have good bioavailability and stability and low toxicity and can thus be safely and effectively used in mammals (e.g. humans, bovines, horses, pigs, dogs, cats, mice, rats and rabbits to name a few).

### EXAMPLES

The examples are described for the purposes of illustration and are not intended to limit the scope of the invention.

Methods of chemistry, protein and peptide biochemistry, cell biology, histology, cellular biology, molecular biology and immunology referred to but not explicitly described in this disclosure and examples are reported in the scientific to literature and are well known to those skilled in the art.

### Example 1

Cell Lines and Chemicals

MC3T3-E1 cells were maintained in Minimum Essential Medium Eagle-modification supplemented with 10% fetal bovine serum (Gibco-BRL) and subcultured as previously described (20). For mineralization, cultures of MC3T3-E1 cells were supplemented with ascorbate (50 μg/ml) and β-glycerolphosphate (β-GP, 10 mM), and the medium was changed every 3 days. These compounds, in addition to ligands, were added to the media once the cultures had reached confluence. MC3T3-637OC stable transfectants were cultured in the same manner and supplemented with active G418 (700 μg/ml). COSP7 cells were cultured in Dulbecco's Modified Eagle's Medium containing 10% FBS and antibiotics. All-trans RA was obtained from Sigma. 4-[E-2-(5,6,7,8-Tetrahydro-5,5,8,8-tetramethyl-2-naphthalenyl)-1-propenyl] benzoic acid (TTNPB), 1,25 VD₃ and 9-cis RA were purchased from BioMol.

### Example 2

Histological Analysis of Mineralization

MC3T3-E1 cells were seeded at 2×10⁴ cells/well into 12-well plates, and medium was changed at three-day intervals for a period of 4 weeks. Matrix calcification within the cultures was measured by histological staining with Alizarin Red S (Sigma). Cells were fixed for 10 min in equal parts of 40% formaldehyde and methanol, rinsed briefly in 50% ethanol followed by a rinse in water. Samples were then stained with Alizarin Red S (2% w/v, pH 4.2) for 2 min, followed by a 30-second wash in acetone and allowed to air dry. A Zeiss SV11 dissection microscope connected to a Sony DXC-950 video camera was used to capture digital images. The extent of mineralization for each culture was determined by calculating the percentage of surface area occupied by Alizarin Red S-stained material using Northern Eclipse image analysis software (Empix Imaging Inc.). All images were captured from the center of the well at low magnification, a field which represents ˜65% of the total surface area of the well.

### Example 3

Analysis of Cell Death and Viability

Cultures of MC3T3-E1 were initiated at sub-confluence (1-2×10⁴ cells/well) in 24-well plates and allowed to reach confluence prior to the addition of ligands, β-GP and ascorbate. Three days following addition of ligand, propidium iodide (PI) dissolved in PBS was added to the culture medium to a final concentration of 2 μg/ml. Cells were incubated in the presence of the dye for 5 minutes, and images were acquired using epifluorescence with a XF35 filter set (Omega Optical) at low magnification (50×). The number of fluorescent-positive cells per microscopic field (3 mm²) was counted using Northern Eclipse imaging software.

TUNEL assays were performed on MC3T3-E1 cultures using a dUTP-fluorescein conjugate according to the manufacturer's instructions (Promega) with minor modifications. Cells were fixed in 4% paraformaldehyde in PBS for 10 minutes, and the TdT incubation was extended to 1.5 hr to improve signal. Prior to mounting, the cell preparations were stained for 15 min with PI at 1 μg/ml. TUNEL positive cells were visualized with epifluorescence using an XF22 filter set (Omega Optical).

Cell viability was measured in MC3T3-E1 cells using the 3-[4,5-dimethylthiazol-2-yl]-2,5-diphenyltetrazolium bromide (MTT) assay in 96-well plates as described in the Roche Molecular Biochemicals product information. Briefly, cells were cultured to confluence, at which time various concentrations and combinations of ligands were added to the cultures followed by incubation for 24 to 60 hr. MTT substrate to a final concentration of 0.5 mg/ml was added to each well and the incubation was extended for a further 4 hr. At this time, cells were solubilized overnight in 10% sodium dodecyl sulfate (SDS) in 0.01 M HCl and absorbance was measured at 595 nm using a 650 nm reference wavelength.

### Example 4

Construction of Dominant-negative RAR- and RXR-EGFP Fusion Genes

Dominant-negative derivatives of RARα and RXRα were constructed using PCR amplification with primers designed to generate C-terminal receptor truncations at amino acid positions 403 and 449 in RARα and RXRα, respectively (21,22). A Bgl II restriction endonuclease site was incorporated into the primers to facilitate cloning and to allow for an in-frame fusion to pEGFP-N1 (Clontech). Internal primers used for truncation of the receptors were, for RARα, 5′- AG ATC TGG GAT CTC CAT CTT CAA TG-3′ and 5′-CAG ATC TCC GAT GAG CTT GAA GAA G-3′ for RXRα. For expression in MC3T3-E1 and COS cell lines, receptor-EGFP fusion constructs were cloned into the mammalian expression plasmid pSG5 (Stratagene). EGFP-N1 was initially subcloned into the pSG5 vector followed by the corresponding truncated receptor to give rise to pSG5-dnRARα EGFP and pSG5-dnRXRα EGFP.

### Example 5

Transient Transfections and Luciferase Assays

Transfection of MC3T3-E1 and COS cell lines were carried out using FuGene6 (Roche Molecular Biochemicals) following the manufacturer's instructions. Cells were seeded either in 6-well or 12-well plates and incubated overnight prior to transfection. DNA-lipid complexes were generated in a two step fashion. First, 3 μl of FuGene6 was added to 97 μl of serum-free medium, and incubated for 5 min at room temperature. After incubation, this mixture was added to 2 μg of DNA and incubated for 15 min at room temperature. This final mixture was used to transfect 4 or 2 wells of a 12- or 6-well plate, respectively.

Analysis of luciferase activity in transiently transfected COS cells using a (RARE)₃ thymidine kinase promoter-luciferase reporter gene was performed as previously described (23) and activity was normalized to that of an internal α-galactosidase expressing control plasmid.

### Example 6

Northern Blotting

Total RNA was isolated with TriPure Isolation Reagent (Roche Molecular Biochemicals) from MC3T3-E1 cultures under mineralizing conditions at various times after culture initiation. RNA samples were separated by electrophoresis of 15 μg aliquots on a 1% agarose-formaldehyde gel. RNA was then transferred to a Hybond-N nylon membrane (Amersham-Pharmacia Biotech) and cross-linked by UV irradiation. Blots were pre-hybridized in Ultrahyb (Ambion) at 45° C. for at least 1 hr. A radiolabeled rat cDNA probe to OC (provided by J. E. Aubin, University of Toronto) was synthesized by random priming. Hybridizations were carried out overnight in Ultrahyb at 42° C. Following hybridization, blots were washed twice with 2×SSC, 0.1% SDS containing buffer for 5 min each at 42° C., followed by two washes in 0.1×SSC, 0.1% SDS for 15 min each at 42° C. and exposed to BioMax X-ray film at −80° C. for 24 hr.

### Example 7

Normal Human Osteoblast Cultures—Effects of RA and RAR

Normal human osteoblast (NHO) were derived from the long bones of a one day old human donor. The cells were purchased from Clonetics (BioWhittaker Company) and cultured according to their protocols with media and serum obtained from Clonetics. Ligands were added to the cell cultures once the cells had reached confluence. Cells were stained with Hoechst 33342, a DNA stain, to allow visualization of the cell nuclei and allow assessment of cell number (the cell nuclei appear white) (FIGS. 7A-C). FIGS. 7D-F the cells were stained with propidium iodide (a membrane impermeant nucleic acid stain) to allow visualization of dead or dying cells. Treatment with all-trans RA appears to increase the number of PI-stained cells suggesting that all-trans RA stimulates cell death in these cultures (dead or dying cells appear white). In 7G, 7I the cells were stained with alizarin red S. The antagonist treated cultures exhibit more alizarin red S staining suggesting that the antagonist promotes bone formation in cultures of normal human osteoblasts (alizarin red S material appears as dark stained material in these figures. In FIGS. 7J-7L the cultures were stained with Von Kossa (a standard histological stain used for examining phosphate deposits, and appears black in these figures), the cultures treated with the antagonist exhibit enhanced Von Kossa staining indicating that the antagonist promotes bone nodule formation of cultures of normal human osteoblasts.

While various embodiments have been described herein in detail, it is understood that variations may be made thereto without affecting the scope of the invention.

References

1. Jones, G., Strugnell, S. A., and DeLuca, H. F. (1998) Physiol Rev 78(4), 1193-231.

2. DeLuca, H. F., and Zierold, C. (1998) Nutr Rev 56(2 Pt 2), S4-10; discussion S 54-75.

3. Aubin, J. E., and Heersche, J. N. M. (1997) in Vitamin D (Feldman, D., Glorieux, F. H., and Pike, J. W., eds), Academic Press, San Diego.

4. Haussler, M. R., Haussler, C. A., Jurutka, P. W., Thompson, P. D., Hsieh, J. C., Remus, L. S., Selznick, S. H., and Whitfield, G. K. (1997) J Endocrinol 154(Suppl), S57-73.

5. Haussler, M. R., Whitfield, G. K., Haussler, C. A., Hsieh, J. C., Thompson, P. D., Selznick, S. H., Dominguez, C. E., and Jurutka, P. W. (1998) J Bone Miner Res 13(3), 325-49.

6. Merke, J., Milde, P., Lewicka, S., Hugel, U., Klaus, G., Mangelsdorf, D. J., Haussler, M. R., Rauterberg, E. W., and Ritz, E. (1989) J Clin Invest 83(6), 1903-15.

7. Milde, P., Merke, J., Ritz, E., Haussler, M. R., and Rauterberg, E. W. (1989) J Histochem Cytochem 37(11), 1609-17.

8. Clemens, T. L., Garrett, K. P., Zhou, X. Y., Pike, J. W., Haussler, M. R., and Dempster, D. W. (1988) Endocrinology 122(4), 1224-30.

9. Berger, U., Wilson, P., McClelland, R. A., Colston, K., Haussler, M. R., Pike, J. W., and Coombes, R. C. (1988) J Clin Endocrinol Metab 67(3), 607-13.

10. Crofts, L. A., Hancock, M. S., Morrison, N. A., and Eisman, J. A. (1998) Proc Natl Acad Sci USA 95(18), 10529-34.

11. Schrader, M., Muller, K. M., Becker-Andre, M., and Carlberg, C. (1994) J Mol Endocrinol 12(3), 327-39.

12. Schrader, M., Bendik, I., Becker-Andre, M., and Carlberg, C. (1993) J Biol Chem 268(24), 17830-6.

13. Li, Y. C., Pirro, A. E., Amling, M., Delling, G., Baron, R., Bronson, R., and Demay, M. B. (1997) Proc Natl Acad Sci USA 94(18), 9831-5.

14. Yoshizawa, T., Handa, Y., Uematsu, Y., Takeda, S., Sekine, K., Yoshibara, Y., Kawakami, T., Arioka, K., Sato, H., Uchiyama, Y., Masushige, S., Fukamizu, A., Matsumoto, T., and Kato, S. (1997) Nat Genet 16(4), 391-6.

15. Underhill, T. M., and Weston, A. D. (1998) Micro. Res. Tech. 43(2), 137-155.

16. McGuire, J., and Lawson, J. P. (1987) Dermatologica 175 Suppl(1), 169-181.

17. Hough, S., L. V., A., Muir, H., Gelderblom, D., Jenkins, G., Kurasi, H., Slatopolsky, E., Bergfeld, M. A., and Teitelbaurn, S. L. (1988) Endocrinol. 122(6), 2933-2939.

18. Chambon, P. (1996) FASEB J. 10, 940-954.

19. Glass, C. K. (1994) Endo. Rev. 15(2), 391-407.

20. Quarles, L. D., Yohay, D. A., Lever, L. W., Caton, R., and Wenstrup, R. J. (1992) J. Bone Min. Res. 7(6), 683-692.

21. Damm, K., Heyman, R. A., Umesono, R. A., and Evans, R. M. (1993) Proc. Natl. Acad. Sci. USA 90, 2989-2993.

22. Feng, X., Peng, Z.-H., Di, W., Li, X.-Y., Rochette-Egly, C., Chambon, P., Voorhees, J. J., and Xiao, J.-H. (1997) Genes & Dev 11, 59-71.

23. Underhill, T. M., Cash, D. E., and Linney, E. (1994) Mol. Endo. 8(3), 274-285.

24. Choi, J. Y., Lee, B. H., Song, K. B., Park, R. W., Kim, I. S., Sohn, K. Y., Jo, J. S., and Ryoo, H. M. (1996) J Cell Biochem 61(4), 609-18.

25. Teng, M., Duong, T. T., Klein, E. S., Pino, M. E., and Chandraratna, R. A. (1996) J Med Chem 39(16), 3035-8.

26. Lian, J. B., Shalhoub, V., Aslam, F., Frenkel, B., Green, J., Hamrah, M., Stein, G. S., and Stein, J. L. (1997) Endocrinology 138(5), 2117-27.

27. Owen, T. A., Aronow, M. S., Barone, L. M., Bettencourt, B., Stein, G. S., and Lian, J. B. (1991) Endocrinology 128(3), 1496-504.

28. Zhang, R., Ducy, P., and Karsenty, G. (1997) J Biol Chem 272(1), 110-6.

29. MacDonald, P. N., Dowd, D. R., Nakajima, S., Galligan, M. A., Reeder, M. C., Haussler, C. A., Ozato, K., and Haussler, M. R. (1993) Mol. Cell. Biol. 13(9), 5907-5917.

30. Raval-Pandya, M., Freedman, L. P., Li, H., and Christakos, S. (1998) Mol Endocrinol 12(9), 1367-79.

31. Pfahl, M. (1993) Endocr Rev 14(5), 651-8.

32. James, S. Y., Williams, M. A., Newland, A. C., and Colston, K. W. (1999) Gen Pharmacol 32(1), 143-54.

33. Fanjul, A., Dawson, M. I., Hobbs, P. D., Jong, L., Cameron, J. F., Harlev, E., Graupner, G., Lu, X.-P., and Pfahl, M. (1994) Nature 372, 107-111.

34. Chen, J. Y., Penco, S., Ostrowski, J., Balaguer, P., Pons, M., Starrett, J. E., Reczek, P., Chambon, P., and Gronemeyer, H. (1995) 14 (1187-1197).

35. Schule, R., Rangarajan, P., Yang, N., Kliewer, S., Ransone, L. J., Bolado, J., Verma, I. M., and Evans, R. M. (1991) Proc Natl Acad Sci USA 88(14), 6092-6.

36. Thompson, P. D., Hsieh, J. C., Whitfield, G. K., Haussler, C. A., Jurutka, P. W., Galligan, M. A., Tillman, J. B., Spindler, S. R., and Haussler, M. R. (1999) Journal of Cellular Biochemistry 75(3), 462-480.

37. Weston, A. D., Rosen, V., Chandraratna, R. A. S., and Underhill, T. M. (2000) J. Cell Biol. in press.

38. Salusky, I. B., and Goodman, W. (1996) Kidney Int Suppl 53, S135-9.

39. Melhus, H., Michaelsson, K., Kindmark, A., Bergstrom, R., Holmberg, L., Mallmin, H., Wolk, A., and Ljunghall, S. (1998) Ann Intern Med 129(10), 770-8.

40. Dong, Y., and E. Canalis. 1995. Insulin-like growth factor (IGF) I and retinoic acid induce the synthesis of IGF-binding protein 5 in rat osteoblastic cells. Endocrinology. 136:2000-2006.

41. Gabbitas, B., and E. Canalis. 1997. Retinoic acid regulates the expression of insulin-like growth factors I and II in osteoblasts. J Cell Phystol. 172:253-264.

42. Heath, J. K., S. B. Rodan, K. Yoon, and G A. Rodan. 1989a. Rat calvarial cell lines immortalized with SV-40 large T antigen: constitutive and retinoic acid-inducible expression of osteoblastic features. Endocrinology. 124:3060-3068.

43. Heath, J. K., S. B. Rodan, K. Yoon, and G. A. Rodan. 1989b. SW-4O large-T immortalization of embryonic bone cells: establishment of osteoblastic clonal cell lines. Connect Tissue Res. 20: 15-21.

44. Heath, J. K., L. J. Suva, K. Yoon, M. Kiledjian, T. J. Martin, and G A. Rodan. 1992. Retinoic acid stimulates transcriptional activity from the alkaline phosphatase promoter in the immortalized rat calvarial cell line, RCT-1. Mol Endocrinol. 6:636-646.

45. Kaji, H., T. Sugirnoto, M. Kanatani, M. Fukase, M. Kumegawa, and K. Chihara 1995. Retinoic acid induces osteoclast-like cell formation by directly acting on hemopoietic blast cells and stimulates osteopontin mRNA expression in isolated osteoclasts. Life Sci. 56:1903-1913.

46. Katagiri, T., A. Yamaguchi, T. Ikeda, S. Yoshiki, J. M. Wozney, V. Rosen, E. A. Wang, H. Tanaka, S. Omura, and T. Suda. 1990. The non-osteogenic mouse pluripotent cell line, C3H1OT1/2, is induced to differentiate into osteoblastic cells by recombinant human bone morphogenetic protein-2. Biochem Biophys Res Commun. 172:295-299.

47. Kirk, M. D., and A. J. Kahn, 1995. Extracellular matrix synthesized by clonal osteogenic cells is osteoinductive in vivo and in vitro: role of transforming growth factor-beta 1 in osteoblast cell-matrix interaction. J Bone Miner Res. 10:1203-1208.

48. Kocijancic, M. 1995. 13-cis-retinoic acid and bone density. Int J Dermatol. 34:733-734. Lafage-Proust, M. H., G. Wesolowski, M. Ernst, G A. Rodan, and S. B. Rodan. 1999. Retinoic acid effects on an SV-40 large T antigen immortalized adult rat bone cell line. J Cell Physiol. 179:267-275.

49. Nakayama, Y., K. Takahashi, S. Noji, K. Muto, K. Nishijima, and S. Taniguchi. 1990. Functional modes of retinoic acid in mouse osteoblastic clone MC3T3-E1, proved as a target cell for retinoic acid. FEBS. 261:93-96.

50. Ng, K. W., P. R. Gummer, V. P. Michelangeli, J. F. Bateman, T. Mascara, W. G. Cole, and Ti. Martin. 1988. Regulation of alkaline phosphatase expression in a neonatal rat clonal calvarial cell strain by retinoic acid. J Bone Miner Res, 3:53-61.

51. Ohishi, K., S. Nishikawa, T. Nagata, N. Yamauchi, H. Shinohara, J. Kido, and H. Ishida. 1995, Physiological concentrations of retinoic acid suppress the osteoblastic differentiation of fetal rat calvaria cells in vitro. Eur J Endocrinol. 133:335-341.

52. Oliva, A., F. Della Ragione, M. Fratta, G. Marrone, R. Palumbo, and V. Zappia. 1993. Effect of retinoic acid on osteocalein gene expression in human osteoblasts. Biochem Biophys Res Commun. 191:908-914.

53. Oreffo, R. O., A. Teti, J. T. Triffitt, M. J. Francis, A. Carano, and A. Z. Zallone. 1988. Effect of vitamin A on bone resorption: evidence for direct stimulation of isolated chicken osteoclasts by retinol and retinoic acid. J Bone Miner Res. 3:203-210.

54. Saneshige, S., H. Mano, K. Tezuka, S. Kakudo, Y. Mori, Y. Honda, A. Itabashi, T. Yamada, K. Miyata, Y. Hakeda, and et al. 1995. Retinoic acid directly stimulates osteoclastic bone resorption and gene expression of cathepsin K/OC-2. Biochem J. 309:721-724.

55. Scheven, B. A., and N. J. Hamilton. 1990. Retinoic acid and 1,25-dihydroxyvitamin D3 stimulate osteoclast formation by different mechanisms. Bone. 11:53-59.

56. Suva, L. J., M. Ernst, and G. A. Rodan. 1991. Retinoic acid increases zif268 early gene expression in rat preosteoblastic cells. Mol Cell Biol. 11:2503-2510.

57. Tobias, J. H., A. Gallagher, and T. J. Chambers. 1994. Intermittent retinoic acid in combination with continuous oestradiol-17 beta increases cancellous bone volume in osteopaenic ovariectomized rats. J Endocrinol. 142:61-67.

58. Togari, A., M. Kondo, M. Arai, and S. Matsumoto. 1991. Effects of retinoic acid on bone formation and resorption in cultured mouse calvaria. Gen Pharmacol. 22:287-292.

59. Wu, B., B. Xu, T. Y. Huang, and J. R. Wang. 1996. [A model of osteoporosis induced by retinoic acid in male Wistar rats]. Yao Hsueh Hsueh Pao. 31:241-245.

60. Zhou, H., R. Q. Hammonds, Jr., D. M. Findlay, P J. Fuller, T. J. Martin, and K. W. Ng. 1991. Retinoic acid modulation of mRNA levels in malignant, nontransformed, and immortalized osteoblasts. J Bone Miner Res. 6:767-777.

1. A pharmaceutical composition for stimulation of osteogenesis, said composition comprising:
a therapeutically effective amount of an RAR antagonist; and
a pharmaceutically acceptable carrier.

2. The composition of claim 1, wherein said RAR antagonist is selected from any chemical that binds to one or more RAR subtype with a Kd of less than 1 micromolar.

3. The composition of claim 2, wherein said RAR antagonist is selected from the group consisting of monofluoro substituted methylchromenes and difluoro substituted methylchromenes.

4. The composition of claim 3, wherein said RAR antagonist is AGN 194301.

5. The composition of claim 2, wherein said RAR, antagonist is selected from the group consisting of AGN 194301, AGN 19309, AGN 190121, AGN 194574, AGN 1931747 AGN 193639, AGN 193676, AGN 193644, SRI 11335, RO41-5253, RO40-6055, CD 2366, BMS 185411, BMS 189453, CD-2665, CD 2019, CD 2781, CD 2665 and CD 271 and mixtures thereof.

6. The composition of claim 1, wherein said composition additionally comprises an agent selected from the group consisting of epidermal growth factor, fibroblast growth factor, platelet derived growth factor, transforming growth factor, parathyroid hormone, leukemia inhibitory factor, insulin-like growth factor, boned morphogenetic protein, osteogenin, sodium fluoride, estrogens, calcitonin, biphosphonates, calcium carbonate, prostaglins, vitamin K and mixtures thereof.

7. The composition of claim 1, wherein said composition stimulates osteogenesis of cells selected from the group consisting of embryonic stem cells, adult stem cells. osteoblastic cells, preosteoblastic cells and skeletal progenitor cells derived from bone, bone marrow or blood.

8. The composition of claim 1, wherein said composition promotes osteoblast differentiation leading to increased mineralization in osteoblastic cells.

9. The composition of claim 8, wherein said composition increases expression of osteocalcin and bone sialoprotein genes in osteoblastic cells.

10. The composition of claim 1, wherein said composition simulates osteogenesis in dedifferentiated cells.

11. The composition of claim 1, wherein said composition is administered locally.

12. The composition of claim 1, wherein said composition is administered systemically.

13. The composition of claim 1, wherein said composition additionally comprises excipients, preservatives, solubilizers, buffering agents, albumin, lubricants, fillers, stabilizers and mixtures thereof.

14. The composition of claim 1, wherein said composition is formulated in a form selected from the group consisting of a liquid solution, liquid emulsion, liquid suspension, coated capsules, pills, tablets, suppositories, lyophilized powders, transdermal patches, lotions and creams.

15. The composition of claim 14, wherein said formulation is provided as a liquid solution, emulsion or suspension encapsulated within a vesicle selected from the group consisting of liposomes, microspheres and nanospheres.

16. The composition of claim 1, wherein said composition is formulated in a controlled release form selected from the group consisting of a biodegradable film, a biodegradable coating and a biodegradable matrix.

17. The composition of claim 16, wherein said film, coating or matrix are applied on prosthetic devices and surgical implants.

18. The composition of claim 17, wherein said composition is applied on the outer surfaces of surgical screws, surgical rods, surgical pins and surgical plates.

19. The composition of claim 1, wherein said composition is formulated and applied onto or embedded within non biodegradable matrices comprising prosthetic devices and surgical implants.

20. The composition of claim 1 wherein said composition is administered for the treatment of metabolic bone diseases and non-metabolic bone diseases.

21. The composition of claim 20, wherein said composition is administered for the treatment of vitamin D toxicity.

22. The composition of claim 20, wherein said composition is administered for the treatment of vitamin A toxicity.

23. The composition of claim 20, wherein said composition is administered for the treatment of bone fractures, bone deformation, spinal deformation, osteosarcoma, myeloma, bone dysplasia, scoliosis, periodontal disease and defects, tooth repair, osteoporosis, arthritis, osteomalcia, fibrous osteitis, renal bone dystrophy and Paget's disease.

24. The composition of claim 1, wherein said composition is administered during bone surgery to promote bone healing.

25. The composition of claim 1, wherein said composition is administered at a dosage range of from about 0.01 mg/kg of body weight to about 300 mg/kg of body weight.

26. The composition of claim 1, wherein said composition is administered for the prevention of vitamin D₃ induced apoptosis in osteoblasts.

27. The use of an RAR antagonist in the manufacture of a medicament for the stimulation of osteogenesis.

28. The use of claim 27, wherein said medicament additionally comprises an agent selected from the group consisting of epidermal growth factor, fibroblast growth factor, platelet derived growth factor, transforming growth factor, parathyroid hormone, leukemia inhibitory factor, insulin-like factor, bone morphogenetic protein, osteogenin, sodium fluoride, estrogens, calcitonin, biphospbonates, calcium carbonate, prostaglins, vitamin K and mixtures thereof.

29. The use of an RAR antagonist in the manufacture of a medicament for the prevention of vitamin D₃ induced apoptosis in osteoblasts.

30. The use of the composition of claim 27, wherein said RAR antagonist is selected from any chemical that binds to one or more RAR subtype with a Kd of less than 1 micromolar.

31. The use of composition of claim 30, wherein said RAR antagonist is selected from the group consisting of monofluoro substituted methylchromenes and difluoro substituted methylchromenes.

32. The use of the composition of claim 31, wherein said RAR antagonist is AGN 194301.

33. The use of the composition of claim 30, wherein said RAR antagonist is selected from the group consisting of AGN 194301, AGN 19309, AGN 190121, AGN 194574, AGN 193174, AGN 193639, AGN 193676, AGN 193644, SRI 11335, RO41-5253, RO40-6055, CD 2366, BMS 185411, BMS 189453, CD-2665, CD 2019, CD 2781, CD 2665 and CD 271 and mixtures thereof.

34. The use of claim 30, wherein said medicament additionally comprises excipients, preservatives, solubilizers, buffering agents, albumin, lubricants, fillers, stabilizers and mixtures thereof.

35. A method for stimulating osteogenesis in a vertebrate having a metabolic bone disease or a non-metabolic bone disease, the method comprising administering to the vertebrate an effective osteogenesis stimulating amount of an RAR antagonist.

36. The method of claim 35, which further comprises administering to said subject one or more agents that promote bone growth or that inhibit bone resorption.

37. The method of claim 36, wherein said agents are selected from the group consisting of bone morphogenetic factors, anti-resorptive agents, osteogenic factors, cartilage-derived morphogenetic proteins, growth hormones, and differentiating factors.

38. The method of claim 37, wherein said agent is selected from the group consisting of epidermal growth factor, fibroblast growth factor, platelet derived growth factor, transforming growth factor, parathyroid hormone, leukemia inhibitory factor, insulin-like growth factor, bone morphogenetic protein, osteogenin, sodium fluoride, estrogens, calcitonin, biphosphonates, calcium carbonate, prostaglandins, vitamin K and mixtures thereof.

39. A method for treating bone associated disorders in a subject, comprising administering to the subject cells selected from the group consisting of embryonic stem cells, adult stem cells, osteoblastic cells, preosteoblastic cells, skeletal progenitor cells derived from bone, bone marrow or blood and mixtures thereof, wherein said cells have been treated in vitro with an effective amount of an RAR antagonist.

40. The method of claim 39, wherein said treated cells are embedded within an implantable matrix comprising a prosthetic device or a surgical implant.

41. A method for the ex vivo stimulation of bone mineralization, said method comprising culturing subject cells selected from the group consisting of embryonic stem cells, adult stem cells, osteoblastic cells, preosteoblastic cells and skeletal progenitor cells derived from bone, bone marrow, or blood, with an effective amount of an RAR antagonist; and incubating said cells for a time sufficient to allow for the promotion of nodule formation.

42. A method for treating or preventing osteoporosis in a human being, comprising administering to said human being in need thereof a therapeutically effective amount of the composition of claim 1.

43. A method of producing bone at a bone defect site in vivo, the method comprising:
implanting into the bone defect site a population of osteoblastic cells or osteoblast progenitor cells which have been cultured in vitro in the presence of a RAR antagonist.

44. The method of claim 43 wherein said cells comprise cells selected from the group consisting of embryonic stem cells and adult stem cells.

45. A method for treating a degenerative joint disease characterized by bone degeneration, the method comprising:
delivering a therapeutically effective amount of a RAR antagonist to a disease site.

46. A method for aiding the attachment of an implantable prosthesis to a bone site and for maintaining the long term stability of the prosthesis in a vertebrate, the method comprising coating selected regions of an implantable prosthesis with a RAR antagonist composition of claim 1 and implanting the coated prosthesis into the bone site, whereby such implantation promotes new bone formation.

47. A pharmaceutical composition for stimulation of osteogenesis, said composition comprising:
a therapeutically effective amount of an RAR antisense oligonucleotide, and
a pharmaceutically acceptable carrier.

48. A method for treating a metabolic or non metabolic bone disease in a patient, said method comprising administering a therapeutically effective amount of a RAR antagonist composition, wherein said composition comprises a RAR antisense oligonucleotide.

49. The method of claim 48, wherein said nucleotide is present in an expression vector and administration to said patient results in the expression of the RAP antisense nucleotide to down regulate or inhibit the transcription of an RAR gene leading to decreased RAR activity.

50. A pharmaceutical composition for inhibiting osteogenesis, said composition comprising a therapeutically effective amount of RAR agonist and a pharmaceutically acceptable carrier therefor.

51. The composition of claim 50, wherein said composition is administered to treat disorders involving increased bone formation.

52. The composition of claim 51, wherein said composition is administered to treat ectopic bone formation.

53. The composition of claim 51, wherein said disorders are selected from the group consisting of osteopetrosis and fibrodysplasia ossificans progressiva.

54. A method for decreasing diseased bone tissue in a mammal, said method comprising administering a therapeutically effective amount of a RAR agonist to said mammal, wherein said RAR agonist inhibits bone mineralization and stimulates apoptosis of diseased bone tissue.

55. A method for stimulating apoptosis in osteoblastic cells, said method comprising administering an effective amount of a RAR agonist to said osteoblastic cells.

56. The use of a RAR agonist in a medicament for the inhibition of osteogenesis.

57. The use of claim 56, wherein said RAR agonist is selected from any naturally occuring or synthetic retinoid compound.

58. The composition of claim 1, wherein said composition is administered for the in vitro or in vivo regulation of VDR transcriptional activity.

59. The composition of claim 21, wherein said composition reverses apoptosis in osteoblastic cells exposed to vitamin D.

60. A method for promoting natural bone formation at a site of skeletal surgery in a vertebrate, the method comprising the steps of delivering a RAR antagonist composition of claim 1 to the site of skeletal surgery whereby such delivery promotes the formation of new bone tissue.

61. A method for inhibiting natural bone formation at a skeletal site in a vertebrate, the method comprising the steps of delivery a RAR agonist composition of claim 49 to the skeletal site whereby such delivery inhibits the formation of new bone tissue.

62. An implantable prosthetic device for repairing bone-associated orthopedic defects and injuries at sites of skeletal surgery, or anomalities in a vertebrate, the device comprising;
a prosthetic implant having a surface region implantable adjacent to or within a bone tissue; and
a RAR antagonist composition disposed on the surface region in an amount sufficient to promote enhanced bone mineralization and bone formation on said surface.

63. The implantable prosthetic device of claim 62, wherein said RAR antagonist composition is disposed within the prosthetic implant.

64. A composition for stimulating osteogenesis in vivo, the composition comprising;
cells selected from the group consisting of embryonic stein cells, adult stem cells, osteoblastic cells, preosteoblastic cells and skeletal progenitor cells derived from bone, bone marrow or blood, wherein said cells have been treated with a therapeutically effective amount of an RAR antagonist; and
a pharmaceutically acceptable carrier.

65. A composition for inhibiting osteogenesis in vivo the composition comprising
cells selected from the group consisting of embryonic stern cells, adult stem cells, osteoblastic cells, preosteoblastic cells and skeletal progenitor cells derived from bone, bone marrow or blood, wherein said cells have been treated with a therapeutically effective amount of an RAR agonist, and
a pharmaceutically acceptable carrier.

66. A method for ex vivo skeletal tissue engineering, said method comprising
culturing a population of cells in the presence of a antagonist composition; and
applying said cells to an implantable matrix and further incubating for a time sufficient for the cells to undergo osteogenesis; wherein the implantable matrix has bone tissue formation incorporating thereon and therein.

67. The method of claim 66, wherein said implantable matrix having bone formation thereon and therein is treated with a RAR agonist composition promote remodeling of the bone tissue.

68. The composition of claim 64 or 65, wherein said cells have additionally been treated with an agent selected from the group consisting of epidermal growth factor, fibroblast growth factor, platelet derived growth factor, transforming growth factor, parathyroid hormone, leukemia inhibitory factor, insulin-like growth factor, bone morphogenetic protein, osteogenin, sodium fluoride, estrogens, calcitonin, biphosphonates, calcium carbonate, prostaglandins, vitamin K and mixtures thereof.

69. The use of claim 27 wherein said stimulation of osteogenesis is in a vertebrate having a metabolic bone disease or a non-metabolic bone disease.

70. The use of claim 69 wherein said medicament further comprises one or more agents that promote bone growth or that inhibit bone resorption.

71. The use of claim 70 wherein said agents are selected from the group consisting of bone morphogenetic factors, anti-resorptive agents, osteogenic factors, cartilage-derived morphogenetic proteins, growth hormones, and differentiating factors.

72. The use of claim 71, wherein said agent is selected from the group consisting of epidermal growth factor, fibroblast growth factor, platelet derived growth factor, transforming growth factor, parathyroid hormone, leukemia inhibitory factor, insulin-like growth factor, bone morphogenetic protein osteogenin, sodium fluoride, estrogens, calcitonin, biphosphonates, calcium carbonate, prostaglandins, vitamin K and mixtures thereof.


SEQUENCE LISTING (2 sequences)

SEQ ID NO: 1 - DNA, 25 bp, Artificial sequence
  Synthetic oligonucleotide primer
agatctgggatctccatcttcaatg

SEQ ID NO: 2 - DNA, 25 bp, Artificial sequence
  Synthetic oligonucleotide primer
cagatctccgatgagcttgaagaag